(19) United States
(12) Patent Application Publication
(10) Pub. No.: US 2013/0060070 A1
(43) Pub. Date: Mar. 7, 2013

(54) METHOD FOR PRODUCING FLUID HYDROCARBONS

(71) Applicants: George W. Huber, Middleton, WI (US); Huiyan Zhang, Nanjing (CN); Torren Carlson, New Castle, DE (US)

(72) Inventors: George W. Huber, Middleton, WI (US); Huiyan Zhang, Nanjing (CN); Torren Carlson, New Castle, DE (US)

(73) Assignee: UNIVERSITY OF MASSACHUSETTS, Boston, MA (US)

(21) Appl. No.: 13/572,736

(22) Filed: Aug. 13, 2012

Related U.S. Application Data
(60) Provisional application No. 61/530,262, filed on Sep. 1, 2011.

(51) Int. Cl. C10G 1/06; C07C 1/22; C10G 1/00

(52) U.S. Cl. 585/242; 585/240; 585/251; 585/252; 585/253; 585/310; 585/319; 585/320; 585/321; 585/324

(57) ABSTRACT

The invention relates to methods for producing fluid hydrocarbon products, and more specifically, to methods for producing fluid hydrocarbon product via catalytic pyrolysis. The reactants comprise solid hydrocarbonaceous materials, and hydrogen or a source of hydrogen (e.g., an alcohol). The products may include specific aromatic compounds (e.g., benzene, toluene, naphthalene, xylene, etc.).

### PRIORITY CLAIM

This application claims priority under 35 U.S.C. §119(e) to U.S. Provisional Application Ser. No. 61/530,262, filed Sep. 1, 2011. The disclosure in this prior application is incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. CBET-0747996 awarded by the National Science Foundation.

### FIELD OF INVENTION

This invention relates to a method for producing fluid hydrocarbons (e.g., biofuel, aromatic compounds, olefin compounds, and the like), and more specifically, to a method for producing fluid hydrocarbons via catalytic pyrolysis.

### BACKGROUND

Environmental issues caused by using fossil fuels, the growing energy demand, and the depletion of petroleum resources have stimulated the development of new sources for the production of renewable liquid fuels. Due to its low cost and abundant availability, solid biomass has been widely studied for the production of liquid fuels. There are several routes for converting solid biomass or its derivatives to liquid fuels. These include aqueous-phase reformation, pyrolysis followed by vapor upgrading, gasification followed by Fischer-Tropsch synthesis, pyrolysis bio-oil hydrogenation, and the conversion of pyrolysis bio-oil or its derivatives to aromatic hydrocarbons over zeolite catalysts. All of these routes involve multiple process steps and tend to be costly.

### SUMMARY

Catalytic pyrolysis, which may include catalytic fast pyrolysis (CFP), is a process by which solid hydrocarbonaceous materials, such as biomass, may be converted into useful hydrocarbon products. This process may comprise a single-step process that uses relatively inexpensive zeolite catalysts. The reactor may be operated at atmospheric pressure. The process may be used to produce a variety of hydrocarbon products including benzene, toluene, xylene, ethylene and propylene. However, a problem with catalytic pyrolysis relates to the need for increasing product yields and providing for more controlled product formation in order to be commercially viable. This invention provides a solution to this problem.

With the present invention, higher yields of desired product formation, lower yields of coke formation, and/or more controlled product formation (e.g., higher production of aromatics and/or olefins relative to other products) may be achieved when particular combinations of reaction conditions and system components are implemented in methods and systems described herein. For example, conditions such as the hydrogen to carbon effective ratio in the feed stream(s), the mass normalized space velocity(ies) (e.g., of the solid hydrocarbonaceous material, the non-solid second reactant, and/or the fluidization fluid), the temperature of the reactor and/or solids separator, the reactor pressure, the heating rate of the feed stream(s), the catalyst to solid hydrocarbonaceous material mass ratio, the residence time of the hydrocarbonaceous material in the reactor, the residence time of the reaction products in the solids separator, and/or the catalyst type (as well as silica to alumina molar ratio for zeolite catalysts) may be controlled to achieve beneficial results.

This invention relates to a method for producing one or more fluid hydrocarbon products from a solid hydrocarbonaceous material comprising: feeding a first reactant comprising the solid hydrocarbonaceous material, and a non-solid second reactant comprising hydrogen or a source of hydrogen, to a reactor; pyrolyzing within the reactor at least a portion of the first reactant under reaction conditions sufficient to produce one or more pyrolysis products; and catalytically reacting at least a portion of the one or more pyrolysis products and at least a portion of the second reactant under reaction conditions sufficient to produce the one or more fluid hydrocarbon products.

The reactor may comprise a continuously stirred tank reactor, a batch reactor, a semi-batch reactor, a fixed bed reactor or a fluidized bed reactor. Fluidized bed reactors may be particularly advantageous.

The first reactant may comprise biomass. The first reactant may comprise plastic waste, recycled plastics, agricultural solid waste, municipal solid waste, food waste, animal waste, carbohydrates, lignocellulosic materials, xylitol, glucose, cellobiose, hemi-cellulose, lignin, sugar cane bagasse, glucose, wood, corn stover, or a mixture of two or more thereof.

The second reactant may comprise molecular hydrogen, or hydrogen that is covalently bonded to a non-hydrogen atom. The second reactant may comprise H₂. The second reactant may comprise an alcohol, ether, ester, carboxylic acid, aldehyde, ketone, hydrocarbon (e.g., alkane, olefin, alkyne, etc.), or a mixture of two or more thereof. The second reactant may comprise methanol, ethanol, propanol, butanol, or a mixture of two or more thereof. In an embodiment, the second reactant may be free of olefins or it may contain an insignificant amount of olefins.

The first reactant and the second reactant may comprise a feed for the reactor wherein the hydrogen to carbon effective ratio for the feed may be in the range from about 0.75 to about 1.5, or from about 0.9 to about 1.5, or from about 1.0 to about 1.4, or from about 1.2 to about 1.3.

The fluidized bed reactor may be operated at a temperature in the range from about 400° C. to about 600° C., or from about 425° C. to about 500° C., or from about 440° C. to about 460° C.

The solid hydrocarbonaceous material may be fed to the reactor at a mass normalized space velocity of less than about 0.9 hour⁻¹, or in the range from about 0.01 hour⁻¹ to about 0.9 hour⁻¹, or in the range from about 0.01 hour⁻¹ to about 0.5 hour⁻¹, or in the range from about 0.1 hour⁻¹ to about 0.9 hour⁻¹, or in the range from about 0.1 hour⁻¹ to about 0.5 hour⁻¹.

The catalytically reacting step may be conducted in the presence of a catalyst. The catalyst may comprise a zeolite catalyst comprising silica and alumina. The silica to alumina molar ratio may be in the range from about 10:1 to about 50:1, or in the range from about 20:1 to about 40:1, or in the range from about 25:1 to about 35:1. The zeolite catalyst may further comprise nickel, platinum, vanadium, palladium, manganese, cobalt, zinc, copper, chromium, gallium, an oxide of one or more thereof, or a mixture of two or more thereof.

The method may be conducted under reaction conditions that minimize coke production. The pyrolysis product may be formed with less than about 30 wt %, or less than about 10 wt %, of the pyrolysis product being coke.

The method may further comprise the step of recovering the one or more fluid hydrocarbon products. The one or more fluid hydrocarbon products may comprise aromatic compounds and/or olefin compounds.

The catalytically reacting step may comprise a dehydration, decarbonylation, decarboxylation, isomerization, oligomerization and/or dehydrogenation reaction.

The pyrolyzing step and the catalytically reacting steps may be carried out in a single vessel. Alternatively, the pyrolyzing step and the catalytically reacting steps may be carried out in separate vessels.

In an embodiment, the one or more fluid hydrocarbon products produced by the inventive method may contain at least about 18 wt %, at least about 20 wt %, at least about 25 wt %, at least about 30 wt %, at least about 35 wt %, at least about 39 wt %, between about 18 wt % and about 40 wt %, between about 18 wt % and about 35 wt %, between about 20 wt % and about 40 wt %, between about 20 wt % and about 35 wt %, between about 25 wt % and about 40 wt %, between about 25 wt % and about 35 wt %, between about 30 wt % and about 40 wt %, or between about 30 wt % and about 35 wt % aromatic compounds.

In an embodiment, the invention relates to a method for producing one or more fluid hydrocarbon products from a solid hydrocarbonaceous material comprising: feeding a first reactant comprising the solid hydrocarbonaceous material and a second, non-solid reactant comprising hydrogen to a fluidized bed reactor such that the hydrogen to carbon effective ratio of the feed is between about 0.75 and about 1.5; pyrolyzing within the fluidized bed reactor at least a portion of the first reactant under reaction conditions sufficient to produce one or more pyrolysis products; and catalytically reacting at least a portion of the one or more pyrolysis products and/or at least a portion of the second reactant under reaction conditions sufficient to produce the one or more fluid hydrocarbon products.

In an embodiment, the invention relates to a method for producing one or more fluid hydrocarbon products from a solid hydrocarbonaceous material comprising: feeding a first reactant comprising the solid hydrocarbonaceous material and a second, non-solid reactant comprising hydrogen to a fluidized bed reactor; pyrolyzing within the fluidized bed reactor at least a portion of the first reactant under reaction conditions sufficient to produce one or more pyrolysis products, wherein the fluidized bed reactor has a temperature of about 400° C. to about 600° C.; and catalytically reacting at least a portion of the one or more pyrolysis products and/or at least a portion of the second reactant under reaction conditions sufficient to produce the one or more fluid hydrocarbon products.

In an embodiment, the invention relates to a method for producing one or more fluid hydrocarbon products from a solid hydrocarbonaceous material comprising: feeding a first reactant comprising the solid hydrocarbonaceous material and a second, non-solid reactant comprising hydrogen to a fluidized bed reactor such that the hydrogen to carbon effective ratio of the feed is between about 0.9 and about 1.5; pyrolyzing within the fluidized bed reactor at least a portion of the first reactant under reaction conditions sufficient to produce one or more pyrolysis products; and catalytically reacting at least a portion of the one or more pyrolysis products and/or at least a portion of the second reactant under reaction conditions sufficient to produce the one or more fluid hydrocarbon products.

In an embodiment, the invention relates to a method for producing one or more fluid hydrocarbon products from a solid hydrocarbonaceous material comprising: feeding a first reactant comprising the solid hydrocarbonaceous material at a mass normalized space velocity of less than about 0.9 hour⁻¹ and a second, non-solid reactant comprising an alcohol to a reactor; pyrolyzing within the reactor at least a portion of the first reactant under reaction conditions sufficient to produce one or more pyrolysis products; and catalytically reacting at least a portion of the one or more pyrolysis products and/or at least a portion of the second reactant under reaction conditions sufficient to produce the one or more fluid hydrocarbon products.

In an embodiment, the invention relates to a method for producing one or more fluid hydrocarbon products from a solid hydrocarbonaceous material comprising: providing a first reactant comprising the solid hydrocarbonaceous material, a second, non-solid reactant comprising an alcohol, and a zeolite catalyst comprising silica and alumina with a silica to alumina molar ratio of from about 10:1 to about 50:1, in a reactor; pyrolyzing within the reactor at least a portion of the first reactant under reaction conditions sufficient to produce one or more pyrolysis products; and catalytically reacting at least a portion of the one or more pyrolysis products and/or at least a portion of the second reactant using the catalyst under reaction conditions sufficient to selectively produce said one or more fluid hydrocarbon products and to minimize coke production.

In an embodiment, the invention relates to a fluid hydrocarbon product which comprises a fluid portion of a reaction product of a first reactant comprising a solid hydrocarbonaceous material and a second, non-solid reactant comprising hydrogen, wherein the mass yield of the aromatic compounds in the fluid hydrocarbon product is at least about 18% by weight.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of this invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B are schematic diagrams of catalytic pyrolysis processes, according to some embodiments;

FIG. 2 is an exemplary schematic diagram of a catalytic pyrolysis process;

FIGS. 3A-3B are exemplary plots of carbon yield as a function of temperature;

FIGS. 4A-4B are exemplary plots of carbon yield as a function of weight hourly space velocity;

FIGS. 5A-5B are exemplary plots of carbon yield as a function of temperature;

FIGS. 6A-6B are, according to some embodiments, plots of carbon yield as a function of weight hourly space velocity;

FIGS. 7A-7B are exemplary plots of carbon yield as a function of hydrogen to carbon effective ratio;

FIGS. 8A-8B are plots of carbon selectivity as a function of hydrogen to carbon effective ratio, according to some embodiments;

FIGS. 9A-9B are, according to some embodiments, plots of carbon yield as a function of hydrogen to carbon effective ratio;

FIGS. 10A-10B are exemplary plots of carbon selectivity as a function of hydrogen to carbon effective ratio;

FIGS. 11A-11B are plots of carbon yield as a function of weight hourly space velocity, according to some embodiments;

FIGS. 12A-12B are exemplary plots of carbon selectivity as a function of weight hourly space velocity;

FIGS. 13A-13H are exemplary mass spectra of chemical process products;

FIG. 14 illustrates the carbon yields of various reaction products, according to one set of embodiments; and

FIG. 15 is an exemplary plot of petrochemical yield as a function of hydrogen to carbon effective ratio.

### DETAILED DESCRIPTION

All ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The transitional words or phrases, such as “comprising,” “including,” “carrying,” “having,” “containing,” “involving,” “holding,” and the like, are to be understood to be open-ended, i.e., to mean including but not limited to.

The terms “pyrolysis” and “pyrolyzing” refer to the transformation of a material (e.g., a solid hydrocarbonaceous material) into one or more other materials (e.g., volatile organic compounds, gases, coke, etc.) by heat, without oxygen or other oxidants or without significant amounts of oxygen or other oxidants, and with or without the use of a catalyst.

The term “catalytic pyrolysis” refers to pyrolysis performed in the presence of a catalyst.

The ratio of hydrogen to carbon in the feed for the inventive method may be adjusted to enhance the amount of a desired product (e.g., aromatic and/or olefin compounds) produced by that method. This ratio may be referred to as the “hydrogen to carbon effective ratio.” The hydrogen to carbon effective ratio, or “H/C_{eff} ratio,” may be calculated using the formula

$$\frac{H}{{C}_{eff}} = \frac{H - \text{?} O - N - \text{?} S}{C} \\ \text{?} \text{indicates text missing or illegible when filed} \qquad [ 1 ]$$

where H, C, O, N, and S are the moles of hydrogen, carbon, oxygen, nitrogen, and sulfur, respectively. One of ordinary skill in the art would be capable of determining the hydrogen to carbon effective ratio for a given feed via elemental analysis of the feed and use of Equation [1]. When determining the hydrogen to carbon effective ratio of a feed, the catalyst composition is not considered. For example, when zeolite catalysts (which generally contain oxygen) are employed, the oxygen within the zeolite catalyst is not considered when determining the hydrogen to carbon effective ratio of the feed. When determining the effective hydrogen to carbon effective ratio of a feed, any fluidization fluid that may be used is not considered.

The terms “aromatics” or “aromatic compound” refer to a hydrocarbon compound or compounds comprising one or more aromatic groups such as, for example, single aromatic ring systems (e.g., benzyl, phenyl, etc.) and/or fused polycyclic aromatic ring systems (e.g. naphthyl, 1,2,3,4-tetrahydronaphthyl, etc.). Examples of aromatic compounds include, but are not limited to, benzene, toluene, indane, indene, 2-ethyl toluene, 3-ethyl toluene, 4-ethyl toluene, trimethyl benzene (e.g., 1,3,5-trimethyl benzene, 1,2,4-trimethyl benzene, 1,2,3-trimethyl benzene, etc.), ethylbenzene, styrene, cumene, methylbenzene, propylbenzene, xylenes (e.g., p-xylene, m-xylene, o-xylene, etc.), naphthalene, methyl-naphthalene (e.g., 1-methyl naphthalene, anthracene, 9.10-dimethylanthracene, pyrene, phenanthrene, dimethyl-naphthalene (e.g., 1,5-dimethylnaphthalene, 1,6-dimethylnaphthalene, 2,5-dimethylnaphthalene, etc.), ethyl-naphthalene, hydrindene, methyl-hydrindene, and dymethyl-hydrindene. Single ring and/or higher ring aromatics may be produced in some embodiments.

The term “petrochemicals” is used herein to refer to chemicals, chemical precursors, chemical intermediates, and the like, traditionally derived from petroleum sources. Petrochemicals include paraffins, olefins, aromatic compounds, and the like. For purposes of this application, when these materials are derived from biomass, as well as other non-petroleum sources (e.g., recycled plastics, municipal solid waste, sugar cane bagasse, wood, etc.), the term petrochemicals may be employed despite the fact that the chemicals, chemical precursors, chemical intermediates, and the like, may not be derived directly from petroleum.

The term “biomass” refers to living and recently dead biological material. In accordance with the inventive method, biomass may be converted, for example, to liquid fuel (e.g., biofuel or biodiesel) or to other fluid hydrocarbon products. Biomass may include trees (e.g., wood) as well as other vegetation; agricultural products and waste (e.g., corn, fruit, garbage, silage, etc.); algae and other marine plants; metabolic wastes (e.g., manure, sewage); and cellulosic urban waste. Biomass may be considered as comprising material that recently participated in the carbon cycle so that the release of carbon in a combustion process may result in no net increase averaged over a reasonably short period of time. For this reason, peat, lignite, coal, shale oil or petroleum may not be considered as being biomass as they contain carbon that may not have participated in the carbon cycle for a long time and, as such, their combustion may result in a net increase in atmospheric carbon dioxide. The term biomass may refer to plant matter grown for use as biofuel, but may also includes plant or animal matter used for production of fibers, chemicals, heat, and the like. Biomass may also include biodegradable waste or byproducts that can be burnt as fuel or converted to chemicals. These may include municipal waste, green waste (the biodegradable waste comprised of garden or park waste such as grass or flower cuttings, hedge trimmings, and the like), byproducts of farming including animal manures, food processing wastes, sewage sludge, black liquor from wood pulp or algae, and the like. Biomass may be derived from plants, including miscanthus, spurge, sunflower, switchgrass, hemp, corn (maize), poplar, willow, sugarcane, and oil palm (palm oil), and the like. Biomass may be derived from roots, stems, leaves, seed husks, fruits, and the like. The particular plant or other biomass source used may not be important to the fluid hydrocarbon product produced in accordance with the inventive method, although the processing of the biomass may vary according to the needs of the reactor and the form of the biomass.

The inventive method may involve feeding a first reactant comprising a solid hydrocarbonaceous material and a non-solid second reactant comprising hydrogen or a source of hydrogen to a reactor. The solid hydrocarbonaceous material may comprise, for example, solid biomass and/or various other solid hydrocarbonaceous materials. The second reactant may comprise molecular hydrogen or hydrogen that is covalently bonded to a non-hydrogen atom. The second reactant may comprise H₂. The second reactant may comprise one or more alcohols, aldehydes, ketones, ethers, esters, carboxylic acids, hydrocarbons (e.g., alkanes, olefins, alkynes, etc.), or a mixture of two or more thereof. At least a portion of the first reactant may be pyrolyzed under reaction conditions sufficient to produce one or more pyrolysis products. At least a portion of the pyrolysis products and at least a portion of the second reactant may be catalytically reacted under sufficient conditions to produce one or more fluid hydrocarbon products. The reactor may comprise a continuously stirred tank reactor, a batch reactor, a semi-batch reactor, a fixed bed reactor, or a fluidized bed reactor. Advantageously, the reactor may comprise a fluidized bed reactor. The catalytic reaction step may be achieved by co-feeding a heterogeneous catalyst with the first and/or second reactants. The catalyst may be fed separately. Part of the catalyst may be fed with either or both of the reactants, and part of the catalyst may be fed separately. The ratio of the first reactant to the second reactant in the feed may be selected to achieve a desired hydrogen to carbon effective ratio for the feed. The hydrogen to carbon effective ratio may be selected, for example, to enhance the amount of aromatic compounds present in the fluid hydrocarbon products produced by the inventive method.

The inventive method may be used for the production of fluid (e.g., a liquid, a supercritical fluid, and/or a gas) hydrocarbon products such as aromatic compounds (e.g., benzene, toluene, naphthalene, xylene, etc.) and olefins (e.g., ethene, propene, butene, etc.) via a catalytic pyrolysis processes (e.g., catalytic fast pyrolysis). The fluid hydrocarbon products, or a portion thereof, may be liquids at standard ambient temperature and pressure (SATP—i.e. 25° C. and 100 kPa absolute pressure). The first and second reactants may be pyrolyzed at intermediate temperatures (for example, in the range from about 400° C. and about 600° C.), compared to temperatures typically used in the prior art. The pyrolysis step may be conducted for an effective amount of time to produce discrete, identifiable fluid hydrocarbon products. The inventive method may involve heating a mixture of the first and second reactants (and optionally the catalyst) to the reaction temperature at relatively high heating rates (e.g., greater than about 50° C. per second) as discussed below.

The inventive method may involve the use of specialized catalysts. For example, zeolite catalysts containing silica and alumina may be used. The catalyst may, in some cases, be formed of or comprise relatively small particles, which may be agglomerated. The composition fed to the pyrolysis reactor may have a relatively high catalyst to hydrocarbonaceous material mass ratio (e.g., from about 2:1 to about 20:1, or from about 5:1 to about 20:1).

The inventive method may comprise a single-stage method for the pyrolysis of solid hydrocarbonaceous materials. This method may comprise providing or using a single-stage pyrolysis apparatus. A single-stage pyrolysis apparatus may be one in which pyrolysis and subsequent catalytic reactions are carried out in a single vessel. The single-stage pyrolysis apparatus may comprise a continuously stirred tank reactor, a bath reactor, a semi-batch reactor, a fixed bed reactor or a fluidized bed reactor. Multi-stage apparatuses may also be used for the production of fluid hydrocarbon products in accordance with the invention.

The first reactant may comprise a solid hydrocarbonaceous material. The first reactant may further comprise one or more liquids, and/or gasses. The solids may be of any suitable size. In some cases, it may be advantageous to use hydrocarbonaceous solids with relatively small particle sizes. Small-particle solids may, in some instances, react more quickly than larger solids due to their relatively higher surface area to volume ratios compared to larger solids. In addition, small particle sizes may allow for more efficient heat transfer within each particle and/or within the reactor volume. This may prevent or reduce the formation of undesired reaction products. Moreover, small particle sizes may provide for increased solid-gas and solid-solid contact, leading to improved heat and mass transfer. In some embodiments, the average size of the solid hydrocarbonaceous material is may be less than about 5 mm, less than about 2 mm, less than about 1 mm, less than about 500 microns, less than about 60 mesh (250 microns), less than about 100 mesh (149 microns), less than about 140 mesh (105 microns), less than about 170 mesh (88 microns), less than about 200 mesh (74 microns), less than about 270 mesh (53 microns), or less than about 400 mesh (37 microns), or smaller.

In some cases, it may be desirable to employ feed material with an average particle size above a minimum amount in order to reduce the pressure required to pass the solid hydrocarbonaceous feed material through the reactor. For example, in some cases, it may be desirable to use solid hydrocarbonaceous material with an average particle size of at least about 400 mesh (37 microns), at least about 270 mesh (53 microns), at least about 200 mesh (74 microns), at least about 170 mesh (88 microns), at least about 140 mesh (105 microns), at least about 100 mesh (149 microns), at least about 60 mesh (250 microns), at least about 500 microns, a least about 1 mm, at least about 2 mm, at least about 5 mm, or higher.

The solid hydrocarbonaceous material may comprise biomass. The solid hydrocarbonaceous material may comprise plastic waste, recycled plastics, agricultural and/or municipal solid waste, food waste, animal waste, carbohydrates, lignocellulosic materials (e.g., wood chips or shavings), or a mixture of two or more thereof. The solid hydrocarbonaceous material may comprise xylitol, glucose, cellobiose, cellulose, hemi-cellulose, lignin, or a mixture of two or more thereof. The solid hydrocarbonaceous material may comprise sugar cane bagasse, glucose, wood, corn stover, or a mixture of two or more thereof. The solid hydrocarbonaceous material may comprise wood.

Biomass pyrolysis liquid or bio-oil may be formed during the pyrolyzing step of the inventive method. Biomass pyrolysis liquid may be dark brown and may approximate to biomass in elemental composition. It may be composed of a very complex mixture of oxygenated hydrocarbons with an appreciable proportion of water from both the original moisture and reaction product. Compositionally, biomass pyrolysis oil may vary with the type of biomass, but is known to contain oxygenated low molecular weight alcohols (e.g., furfuryl alcohol), aldehydes (aromatic aldehydes), ketones (furanone), phenols (methoxy phenols) and water. Solid char may also be present, suspended in the oil. The liquid may be formed by rapidly quenching the intermediate products of flash pyrolysis of hemicellulose, cellulose and lignin in the biomass. Chemically, the oil may contain several hundred different chemicals in widely varying proportions, ranging from formaldehyde and acetic acid to complex high molecular weight phenols, anhydrosugars and other oligosaccharides. It may have a distinctive odor from low molecular weight aldehydes and acids, and may be acidic with a pH of about 1.5 to about 3.8, and can be an irritant.

The non-solid second reactant composition may comprise hydrogen or a source of hydrogen. The second reactant may comprise H₂. The second reactant may comprise a source of hydrogen wherein the hydrogen is covalently bonded to another non-hydrogen atom (e.g., an oxygen or a carbon atom). The source of hydrogen may comprise an alcohol, ether, ester, carboxylic acid, aldehyde, ketone, hydrocarbon (e.g., alkane, olefin, alkyne), or a mixture of two or more thereof. The alcohols may include monols such as methanol, ethanol, propanol, and/or butanol, as well as diols, such as ethylene glycol, etc. The alkanes may include methane, ethane, propane, butane, etc. The olefins or alkenes may include ethylene, propylene, butylenes, etc. The alkynes may include acetylene, propyne, butyne, etc. The esters may include methyl acetate, ethyl acetate, propyl acetate, butyl acetate, methyl propionate, ethyl propionate, propyl propionate, methyl butyrate, ethyl butyrate, etc. The ethers may include dimethyl ether, methyl-t-butyl ether, methyl-t-amyl ether, diethyl ether, etc. The carboxylic acids may include acetic acid, propionic acid, butyric acid, fatty acids, etc. The aldehydes may include acetaldehyde, propionaldehyde, benzaldehyde, etc. The ketones may include acetone, methyl ethyl ketone, etc. The alcohols, including methanol, may be particularly useful. In an embodiment, at least about 50 wt %, or at least about 75 wt %, or at least about 90 wt %, or at least about 95 wt %, or at least about 99 wt % of the second reactant may comprise compounds having carbon numbers of less than about C₁₀, or less than about C₅, or less than about C₃.

The residence time of the catalyst in the reactor may be defined as the volume of the reactor filled with catalyst divided by the volumetric flow rate of the catalyst through the reactor. For example if a 3 liter reactor contains 2 liters of catalyst and a flow of 0.4 liters per minute of catalyst is fed through the reactor, ie both fed and removed, the catalyst residence time will be 2/0.4 minutes, or 5 minutes.

Contact time may be defined as the residence time of a material in a reactor or other device, when measured or calculated under standard conditions of temperature and pressure (i.e., 0° C. and 100 kPa absolute pressure). For example, a 2 liter reactor to which is fed 3 standard liters per minute of gas has a contact time of ⅔ minute, or 40 seconds for that gas. For a chemical reaction, contact time or residence time is based on the volume of the reactor where substantial reaction is occurring; and would exclude volume where substantially no reaction is occurring such as an inlet or an exhaust conduit. For catalyzed reactions, the volume of a reaction chamber is the volume where catalyst is present.

The term “conversion of a reactant” may refer to the reactant mole or mass change between a material flowing into a reactor and a material flowing out of the reactor divided by the moles or mass of reactant in the material flowing into the reactor. For example, if 100 g of ethylene are fed to a reactor and 30 g of ethylene are flowing out of the reactor, the conversion is [(100−30)/100]=70% conversion of ethylene.

The term “fluid” may refer to a gas, a liquid, a mixture of a gas and a liquid, or a gas or a liquid containing dispersed solids, liquid droplets and/or gaseous bubbles. The terms “gas” and “vapor” have the same meaning and are sometimes used interchangeably. In some embodiments, it may be advantageous to control the residence time of the fluidization fluid in the reactor. The fluidization residence time of the fluidization fluid is defined as the volume of the reactor divided by the volumetric flow rate of the fluidization fluid under process conditions of temperature and pressure.

The term “fluidized bed reactor” may be used to refer to reactors comprising a vessel that contains a granular solid material (e.g., silica particles, catalyst particles, etc.), in which a fluid (e.g., a gas or a liquid) is passed through the granular solid material at velocities sufficiently high as to suspend the solid material and cause it to behave as though it were a fluid. The term “circulating fluidized bed reactor” may be used to refer to fluidized bed reactors in which the granular solid material is passed out of the reactor, circulated through a line in fluid communication with the reactor, and recycled back into the reactor.

Bubbling fluidized bed reactors and turbulent fluidized bed reactors may be used. In bubbling fluidized bed reactors, the fluid stream used to fluidize the granular solid material may be operated at a sufficiently low flow rate such that bubbles and voids may be observed within the volume of the fluidized bed during operation. In turbulent fluidized bed reactors, the flow rate of the fluidizing stream may be higher than that employed in a bubbling fluidized bed reactor, and hence, bubbles and voids may not be observed within the volume of the fluidized bed during operation. Examples of fluidized bed reactors, circulating fluidized bed reactors, bubbling and turbulent fluidized bed reactors are described in Kirk-Othmer Encyclopedia of Chemical Technology (online), Vol. 11, Hoboken, N.J.: Wiley Interscience, 2001, pages 791-825, these pages being incorporated herein by reference.

The terms “olefin” or “olefin compound” (a.k.a. “alkenes”) may be used to refer to any unsaturated hydrocarbon containing one or more pairs of carbon atoms linked by a double bond. Olefins may include both cyclic and acyclic (aliphatic) olefins, in which the double bond is located between carbon atoms forming part of a cyclic (closed-ring) or of an open-chain grouping, respectively. In addition, olefins may include any suitable number of double bonds (e.g., monoolefins, diolefins, triolefins, etc.). Examples of olefin compounds may include ethene, propene, allene (propadiene), 1-butene, 2-butene, isobutene (2 methyl propene), butadiene, and isoprene, among others. Examples of cyclic olefins may include cyclopentene, cyclohexane, cycloheptene, among others. Aromatic compounds such as toluene are not considered olefins; however, olefins that include aromatic moieties are considered olefins, for example, benzyl acrylate or styrene.

Pore size relates to the size of a molecule or atom that can penetrate into the pores of a material. As used herein, the term “pore size” for zeolites and similar catalyst compositions refers to the Norman radii adjusted pore size. Determination of Norman radii adjusted pore size is described, for example, in Cook, M.; Conner, W. C., “How big are the pores of zeolites?” Proceedings of the International Zeolite Conference, 12th, Baltimore, Jul. 5-10, 1998; (1999), 1, pp 409-414, which is incorporated herein by reference. As a specific exemplary calculation, the atomic radii for ZSM-5 pores are about 5.5-5.6 Angstroms, as measured by x-ray diffraction. In order to adjust for the repulsive effects between the oxygen atoms in the catalyst, Cook and Conner have shown that the Norman adjusted radii are 0.7 Angstroms larger than the atomic radii (about 6.2-6.3 Angstroms).

One of ordinary skill in the art will understand how to determine the pore size (e.g., minimum pore size, average of minimum pore sizes) in a catalyst. For example, x-ray diffraction (XRD) may be used to determine atomic coordinates. XRD techniques for the determination of pore size are described, for example, in Pecharsky, V. K. et at, “Fundamentals of Powder Diffraction and Structural Characterization of Materials,” Springer Science+Business Media, Inc., New York, 2005, incorporated herein by reference in its entirety. Other techniques that may be useful in determining pore sizes (e.g., zeolite pore sizes) may include, for example, helium pycnometry or low pressure argon adsorption techniques. These and other techniques are described in Magee, J. S. et at, “Fluid Catalytic Cracking: Science and Technology,” Elsevier Publishing Company, Jul. 1, 1993, pp. 185-195, which is incorporated herein by reference in its entirety. Pore sizes of mesoporous catalysts may be determined using, for example, nitrogen adsorption techniques, as described in Gregg, S. J. at al, “Adsorption, Surface Area and Porosity,” 2nd Ed., Academic Press Inc., New York, 1982 and Rouquerol, F. et al, “Adsorption by powders and porous materials. Principles, Methodology and Applications,” Academic Press Inc., New York, 1998, both of which are incorporated herein by reference.

In some embodiments, a screening method may be used to select catalysts with appropriate pore sizes for the conversion of specific pyrolysis product molecules. The screening method may comprise determining the size of pyrolysis product molecules desired to be catalytically reacted (e.g., the molecule kinetic diameters of the pyrolysis product molecules). One of ordinary skill in the art may calculate, for example, the kinetic diameter of a given molecule. The type of catalyst may then be chosen such that the pores of the catalyst (e.g., Norman adjusted minimum radii) are sufficiently large to allow the pyrolysis product molecules to diffuse into and/or react with the catalyst. In some embodiments, the catalysts may be chosen such that their pore sizes are sufficiently small to prevent entry and/or reaction of pyrolysis products whose reaction would be undesirable.

The catalyst may comprise any catalyst suitable for conducting the catalytically reacting step of the inventive method. The catalyst may be used to lower the activation energy (increase the rate) of the reaction conducted in the catalytically reacting step and/or improve the distribution of products or intermediates during the reaction (for example, a shape selective catalyst). Examples of reactions that can be catalyzed include: dehydration, dehydrogenation, isomerization, hydrogen transfer, aromatization, decarbonylation, decarboxylation, aldol condensation, and combinations thereof. The catalyst components may be acidic, neutral or basic.

The inventive method may comprise a catalytic fast pyrolysis (CFP) process. For catalytic fast pyrolysis processes, particularly advantageous catalysts may include those containing internal porosity selected according to pore size (e.g., mesoporous and pore sizes typically associated with zeolites), e.g., average pore sizes of less than about 100 Angstroms, less than about 50 Angstroms, less than about 20 Angstroms, less than about 10 Angstroms, less than about 5 Angstroms, or smaller. In some embodiments, catalysts with average pore sizes of from about 5 Angstroms to about 100 Angstroms may be used. In some embodiments, catalysts with average pore sizes of between about 5.5 Angstroms and about 6.5 Angstroms, or between about 5.9 Angstroms and about 6.3 Angstroms may be used. In some cases, catalysts with average pore sizes of between about 7 Angstroms and about 8 Angstroms, or between about 7.2 Angstroms and about 7.8 Angstroms may be used.

In some embodiments of CFP, the catalyst may be selected from naturally occurring zeolites, synthetic zeolites and combinations thereof. The catalyst may be a ZSM-5 zeolite catalyst. The catalyst may comprise acidic sites. Other zeolite catalysts that may be used may include ferrierite, zeolite Y, zeolite beta, mordenite, MCM-22, ZSM-23, ZSM-57, SUZ-4, EU-1, ZSM-11, (S)A1P0-31, SSZ-23, and the like. Non-zeolite catalysts may be used; for example, WOx/ZrO2, aluminum phosphates, etc. The catalyst may comprise a metal and/or a metal oxide. Suitable metals and/or oxides may include, for example, nickel, palladium, platinum, titanium, vanadium, chromium, manganese, iron, cobalt, zinc, copper, gallium, and/or any of their oxides, among others. In some cases promoter elements selected from the rare earth elements, ie elements 57-71, cerium, zirconium or their oxides, or combinations of these may be included to modify the activity, structure and/or stability of the catalyst. In addition, in some cases, properties of the catalysts (e.g., pore structure, type and/or number of acid sites, etc.) may be chosen to selectively produce a desired product.

Catalysts for other processes, such as alkylation of olefins are well-known and can be selected for the processes described herein.

In some instances, it is beneficial to control the residence time of the reactants (e.g., the solid hydrocarbonaceous material and/or a non-solid reactant) and catalyst(s) in a reactor and/or under a defined set of reaction conditions (i.e. conditions under which the reactants may undergo pyrolysis or catalysis in a given reactor system).

The term “overall residence time” refers to the volume of a reactor or device or specific portion of a reactor or device divided by the exit flow of all gases out of the reactor or device including fluidization gas, products, and impurities, measured or calculated at the average temperature of the reactor or device and the exit pressure of the reactor or device.

The term “reactant residence time” of a reactant in the reactor is defined as the amount of time the reactant spends in the reactor. Residence time may be based on the feed rate of reactant and is independent of rate of reaction. The reactant residence time of the reactants in a reactor may be calculated using different methods depending upon the type of reactor being used. For gaseous reactants, where flow rate into the reactor is known, this is typically a simple calculation. In the case of solid reactants in which the reactor comprises a packed bed reactor into which only reactants are continuously fed (i.e. no carrier or fluidizing flow is utilized), the reactant residence time in the reactor may be calculated by dividing the volume of the reactor by the volumetric flow rate of the hydrocarbonaceous material and fluid hydrocarbon product exiting the reactor.

In cases where the reaction takes place in a reactor that is closed to the flow of mass during operation (e.g., a batch reactor), the batch residence time of the reactants in such may be reactor is defined as the amount of time elapsing between the time at which the temperature in the reactor containing the reactants reaches a level sufficient to commence a pyrolysis reaction (e.g., for CFP, typically about 300° C. to about 1000° C. for many typical hydrocarbonaceous feedstock materials) and the time at which the reactor is quenched (e.g., cooled to a temperature below that sufficient to support further pyrolysis—e.g. typically about 300° C. to about 1000° C. for many hydrocarbonaceous feedstock materials).

In some cases, e.g. for certain fluidized bed reactors, the reactor feed stream(s) may include feed stream(s) comprising auxiliary materials (i.e., matter other than solid hydrocarbonaceous materials and/or non-solid reactants). For example, in certain cases where fluidized beds are used as reactors, the feed stream may comprise fluidization fluid(s). In cases where circulating fluidized beds are used, catalyst and fluidization fluid may both be fed, recycled, or fed and recycled to the reactor. In such cases, the reactant residence time of the reactants in the reactor can be determined as the volume of the reactor divided by the volumetric flow rate of the reactants and reaction product gases exiting the reactor as with the packed bed situation described above; however, since the flow rate of the reactants and reaction product gases exiting the reactor may not be convenient to determine directly, the volumetric flow rate of the reactants and reaction product gases exiting the reactor may be estimated by subtracting the feed volumetric flow rate of the auxiliary materials (e.g., fluidization fluid, catalyst, contaminants, etc.) into the reactor from the total volumetric flow rate of the gas stream(s) exiting the reactor.

The term “selectivity” refers to the amount of production of a particular product in comparison to a selection of products. Selectivity to a product may be calculated by dividing the amount of a particular product by the amount of a number of products produced. For example, if 75 grams of aromatics are produced in a reaction and 20 grams of benzene are found in these aromatics, on a mass basis the selectivity to benzene amongst aromatic products is 20/75=26.7%. Selectivity may be calculated on a mass basis, as in the aforementioned example, or it may be calculated on a carbon basis where the selectivity is calculated by dividing the amount of carbon that is found in a particular product by the amount of carbon that is found in a selection of products. Unless specified otherwise, for reactions involving biomass as a reactant, selectivity is on a mass basis. For reactions involving conversion of a specific molecular reactant (ethene for example), selectivity is the percentage (on a mass basis unless specified otherwise) of a selected product divided by all the products produced.

The term “yield” is used herein to refer to the amount of a product flowing out of a reactor divided by the amount of reactant flowing into the reactor, usually expressed as a percentage or fraction. Yields are often calculated on a mass basis, carbon basis, or on the basis of a particular feed component. Mass yield is the mass of a particular product divided by the weight of feed used to prepare that product. For example, if 500 grams of biomass is fed to a reactor and 45 grams of benzene is produced, the mass yield of benzene would be 45/500=9% benzene. Carbon yield is the mass of carbon found in a particular product divided by the mass of carbon in the feed to the reactor. For example, if 500 grams of biomass that contains 40% carbon is reacted to produce 45 g of benzene that contains 92.3% carbon, the carbon yield is [(45*0.923)/(500*0.40)]=20.8%. Carbon yield from biomass is the mass of carbon found in a particular product divided by the mass of carbon fed to the reactor in a particular feed component. For example, if 500 grams of biomass containing 40% carbon and 100 grams of CO2 are reacted to produce 40 g of benzene (containing 92.3% carbon), the carbon yield on biomass is [(40*0.923)/(500*0.40)]=18.5%; note that the mass of CO2 does not enter into the calculation.

The embodiments described herein may also involve chemical process designs used to perform catalytic pyrolysis. In some cases, the processes may involve the use of one or more fluidized bed reactors (e.g., a circulating fluidized bed reactor, turbulent fluidized bed reactor, bubbling fluidized bed reactor, etc.). The process designs described herein may optionally involve specialized handling of the material fed to one or more reactors. For example, in some embodiments, the feed material may be dried, cooled, and/or ground prior to supplying the material to a reactor. Other aspects of the invention relate to product compositions produced using the process designs described herein.

Without being bound to a particular mode of action or order of steps of the overall thermal/catalytic conversion process, catalytic pyrolysis is believed to involve at least partial thermal pyrolysis of hydrocarbonaceous material (e.g., solid biomass such as cellulose) to produce one or more pyrolysis products (e.g., volatile organics, gases, solid coke, etc.) and catalytic reaction of at least a portion of the one or more pyrolysis products using a catalyst under reaction conditions sufficient to produce fluid hydrocarbon products. The catalytic reaction may involve volatile organics entering into a catalyst (e.g., a zeolite catalyst) where they are converted into, for example, hydrocarbons such as aromatics and olefins, in addition to carbon monoxide, carbon dioxide, water, and coke. Inside or upon contact with the catalyst, the biomass-derived species may undergo a series of dehydration, decarbonylation, decarboxylation, isomerization, oligomerization, and dehydrogenation reactions that lead to aromatics, olefins, CO, CO₂ and water.

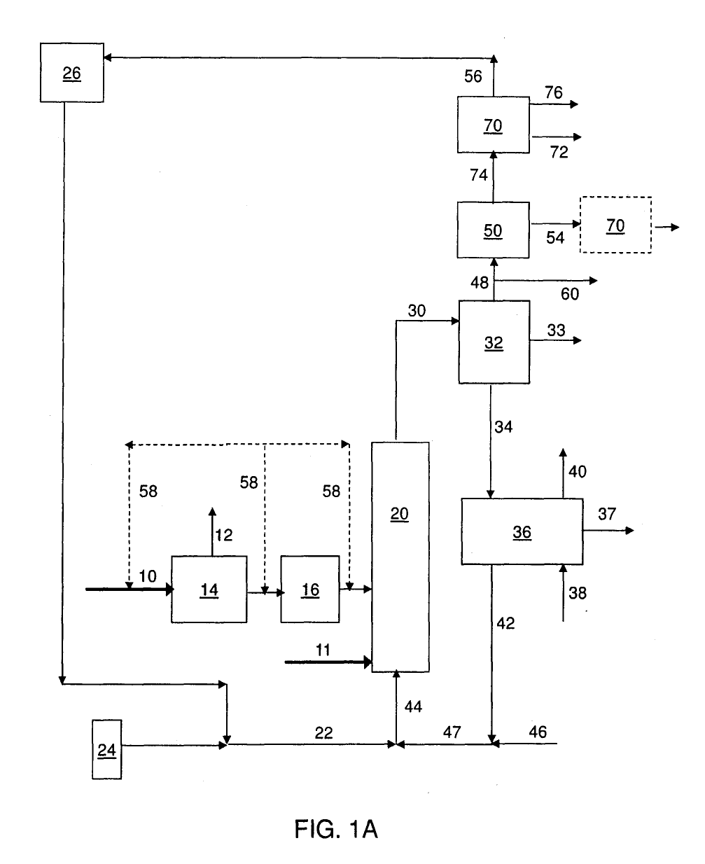

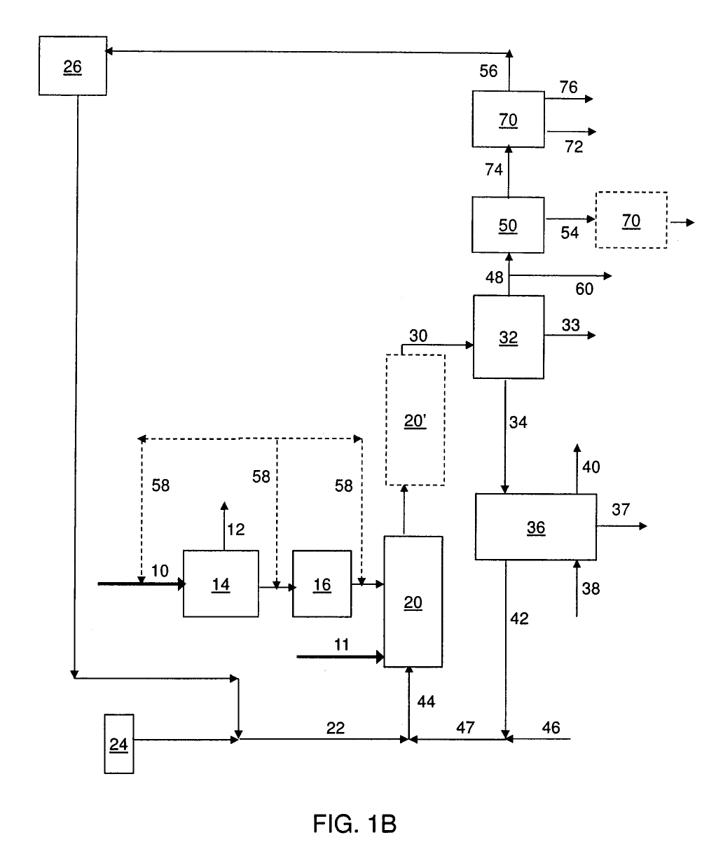

FIG. 1A includes a schematic illustration of an exemplary chemical process design used to perform catalytic pyrolysis, according to one set of embodiments. In some embodiments, such a process may be used to perform catalytic fast pyrolysis. As shown in the illustrative embodiment of FIG. 1A, a feed stream 10 includes a first reactant comprising a solid hydrocarbonaceous material that can be fed to a reactor 20. The solid hydrocarbonaceous material may generally comprise at least carbon and hydrogen. In certain solid hydrocarbonaceous materials (e.g. wood), carbon is the most abundant component by mass, while in others (e.g. glucose) oxygen may be more abundant than carbon. Certain solid hydrocarbonaceous materials may also comprise relatively minor proportions of other elements such as nitrogen and sulfur. Specific, non-limiting examples of solid hydrocarbonaceous materials are provided below.

In FIG. 1A, a second feed stream 11 comprising a non-solid second reactant composition comprising hydrogen or a source of hydrogen, can be fed to reactor 20.

The feed streams to the reactor (including the first reactant feed stream and/or the second reactant feed stream) may be free of olefins, or may contain olefins in an insignificant amount (e.g., such that olefins make up less than about 1 wt %, less than about 0.1 wt %, or less than about 0.01 wt % of the total weight of reactant fed to the reactor). For example, there may be no olefins present within the non-solid second reactant. In other embodiments, however, olefins may be present in one or more reactant feed streams.

The hydrogen or source of hydrogen may be derived from a process stream (e.g., a waste stream) from another process, for example, a fermentation process, a distillation process, etc.

While FIG. 1A includes an independent feed stream 11 for feeding the non-solid second reactant to reactor 20, it should be understood that the invention is not limited to this configuration. For example, in some embodiments, the non-solid second reactant may be mixed with the first reactant comprising the solid hydrocarbonaceous material (e.g., within feed stream 10) prior to entering the reactor, in addition to or in place of feeding the second reactant via independent feed stream 11. In some embodiments, the second reactant may be mixed with a fluidization fluid provided to reactor 20 (e.g., via inlet 44, described in more detail below) prior to entering the reactor, in addition to or in place of feeding the second reactant via independent feed stream 11 and/or mixing the second reactant with the first reactant prior to feeding the mixture to the reactor.

In some embodiments, the feeds of the first and second reactants may be selected to produce a desired hydrogen to carbon effective ratio of the materials within the first and second reactants. In some embodiments, the hydrogen to carbon effective ratio of the feed to the reactor (including both the first reactant comprising the solid hydrocarbonaceous material and the second, non-solid reactant) may be in the range from about 0.75 and about 1.5, or about 0.9 and about 1.5, or about 1.0 and about 1.4, or about 1.2 and about 1.3. A desired hydrogen to carbon effective ratio of a feed may be achieved according to the invention, for example, by adjusting the flow rates of the first and second reactants, or by pre-mixing appropriate amounts of first and second reactants.

In some embodiments, the solid hydrocarbonaceous material feed composition (e.g., in feed stream 10 of FIG. 1A) may comprise a mixture of solid hydrocarbonaceous material and a catalyst. The mixture may comprise, for example, a solid catalyst and a solid hydrocarbonaceous material. In other embodiments, a catalyst may be provided separately from the solid hydrocarbonaceous material (e.g., by co-feeding the catalyst with the second reactant and/or by feeding the catalyst via an independent catalyst inlet). A variety of catalysts may be used, as described in more detail below. For example, in some instances, zeolite catalysts with varying molar ratios of silica to alumina, and/or varying pore sizes, and/or varying catalytically active metals and/or metal oxides, may be used.

In some embodiments, moisture 12 may optionally be removed from the solid hydrocarbonaceous feed composition prior to being fed to the reactor, e.g., by an optional dryer 14. Removal of moisture from the solid hydrocarbonaceous material feed stream may be advantageous for several reasons. For example, the moisture in the feed stream may require additional energy input in order to heat the solid hydrocarbonaceous material to a temperature sufficiently high to achieve pyrolysis. Variations in the moisture content of the solid hydrocarbonaceous feed may lead to difficulties in controlling the temperature of the reactor. In addition, removal of moisture from the solid hydrocarbonaceous feed can reduce or eliminate the need to process the water during later processing steps.

In some embodiments, the solid hydrocarbonaceous feed composition may be dried until the solid hydrocarbonaceous feed composition comprises less than about 10%, less than about 5%, less than about 2%, or less than about 1% water by weight. Suitable equipment capable of removing water from the feed composition is known to those skilled in the art. As an example, in one set of embodiments, the dryer comprises an oven heated to a particular temperature (e.g., at least about 80° C., at least about 100° C., at least about 150° C., or higher) through which the solid hydrocarbonaceous feed composition is continuously, semi-continuously, or periodically passed. In some cases, the dryer may comprise a vacuum chamber into which the solid hydrocarbonaceous feed composition is processed as a batch. Other embodiments of the dryer may combine elevated temperatures with vacuum operation. The dryer may be integrally connected to the reactor or may be provided as a separate unit from the reactor.

In some instances, the particle size of the solid hydrocarbonaceous feed composition may be reduced in an optional grinding system 16 prior to passing the solid hydrocarbonaceous feed to the reactor. In some embodiments, the average diameter of the ground, solid hydrocarbonaceous feed composition exiting the grinding system may comprise no more than about 50%, not more than about 25%, no more than about 10%, no more than about 5%, no more than about 2% of the average diameter of the feed composition fed to the grinding system. Large-particle solid hydrocarbonaceous feed material may be more easily transportable and less messy than small-particle feed material. On the other hand, in some cases it may be advantageous to feed small particles of solid hydrocarbonaceous material to the reactor (as discussed below). The use of a grinding system allows for the transport of large-particle solid hydrocarbonaceous feed between the source and the process, while enabling the feed of small particles to the reactor.

Suitable equipment capable of grinding the solid hydrocarbonaceous feed composition is known to those skilled in the art. For example, the grinding system may comprise an industrial mill (e.g., hammer mill, ball mill, etc.), a unit with blades (e.g., chipper, shredder, etc.), or any other suitable type of grinding system. In some embodiments, the grinding system may comprise a cooling system (e.g., an active cooling systems such as a pumped fluid heat exchanger, a passive cooling system such as one including fins, etc.), which may be used to maintain the solid hydrocarbonaceous feed composition at relatively low temperatures (e.g., ambient temperature) prior to introducing the solid hydrocarbonaceous feed composition to the reactor. The grinding system may be integrally connected to the reactor or may be provided as a separate unit from the reactor. While the grinding step is shown following the drying step in FIG. 1A, the order of these operations may be reversed in some embodiments. In still other embodiments, the drying and grinding steps may be achieved using an integrated unit.

In some cases, grinding and cooling of the solid hydrocarbonaceous material may be achieved using separate units. Cooling of the solid hydrocarbonaceous material may be desirable, for example, to reduce or prevent unwanted decomposition of the solid hydrocarbonaceous feed material prior to passing it to the reactor. In one set of embodiments, the solid hydrocarbonaceous material may be passed to a grinding system to produce a ground solid hydrocarbonaceous material. The ground solid hydrocarbonaceous material may then be passed from the grinding system to a cooling system and cooled. The solid hydrocarbonaceous material may be cooled to a temperature of lower than about 300° C., lower than about 200° C., lower than about 100° C., lower than about 75° C., lower than about 50° C., lower than about 35° C., or lower than about 20° C. prior to introducing the solid hydrocarbonaceous material into the reactor. In embodiments that include the use of a cooling system, the cooling system includes an active cooling unit (e.g., a heat exchanger) capable of lowering the temperature of the solid hydrocarbonaceous material. In some embodiments, two or more of the drier, grinding system, and cooling system may be combined in a single unit. The cooling system may be, in some embodiments, directly integrated with one or more reactors.

As illustrated in FIG. 1A, the solid hydrocarbonaceous material and the non-solid reactant may be transferred to reactor 20. The reactor may be used, in some instances, to perform catalytic pyrolysis of at least a portion of the first reactant comprising the hydrocarbonaceous material under reaction conditions sufficient to produce one or more pyrolysis products. In some embodiments, the reactor may be used to catalytically react at least a portion of the one or more pyrolysis products and/or at least a portion of the second reactant under reaction conditions sufficient to produce the one or more fluid hydrocarbon products. In the illustrative embodiment of FIG. 1A, the reactor comprises any suitable reactor known to those skilled in the art. For example, in some instances, the reactor may comprise a continuously stirred tank reactor (CSTR), a batch reactor, a semi-batch reactor, or a fixed bed catalytic reactor, among others. In some cases, the reactor comprises a fluidized bed reactor, e.g., a circulating fluidized bed reactor. Fluidized bed reactors may, in some cases, provide improved mixing of the catalyst, solid hydrocarbonaceous material, and/or the non-solid reactant during pyrolysis and/or subsequent reactions, which may lead to enhanced control over the reaction products formed. The use of fluidized bed reactors may also lead to improved heat transfer within the reactor. In addition, improved mixing in a fluidized bed reactor may lead to a reduction of the amount of coke adhered to the catalyst, resulting in reduced deactivation of the catalyst in some cases.

The reactor(s) may have any suitable size for performing the processes described herein. For example, the reactor may have a volume between about 0.1-1 L, 1-50 L, 50-100 L, 100-250 L, 250-500 L, 500-1000 L, 1000-5000 L, 5000-10,000 L, or 10,000-50,000 L. In some instances, the reactor may have a volume greater than about 1 L, or in other instances, greater than about 10 L, 50 L, 100 L, 250 L, 500 L, 1,000 L, or 10,000 L. Reactor volumes greater than about 50,000 L are also possible. The reactor may be cylindrical, spherical, or any other suitable shape.

Higher yields of desired product formation, lower yields of coke formation, and/or more controlled product formation (e.g., higher production of aromatics and/or olefins relative to other products) may be achieved when particular combinations of reaction conditions and system components are implemented in methods and systems described herein. For example, conditions such as the hydrogen to carbon effective ratio in the feed stream(s), the mass normalized space velocity(ies) (e.g., of the solid hydrocarbonaceous material, the non-solid second reactant, and/or the fluidization fluid), the temperature of the reactor and/or solids separator, the reactor pressure, the heating rate of the feed stream(s), the catalyst to solid hydrocarbonaceous material mass ratio, the residence time of the hydrocarbonaceous material in the reactor, the residence time of the reaction products in the solids separator, and/or the catalyst type (as well as silica to alumina molar ratio for zeolite catalysts) may be controlled to achieve beneficial results, as described below.

The reactor(s) may be operated at any suitable temperature. In some instances, it may be desirable to operate the reactor(s) at intermediate temperatures, compared to temperatures typically used in many previous catalytic pyrolysis systems. For example, the reactor may be operated at temperatures of between about 400° C. and about 600° C., between about 425° C. and about 500° C., or between about 440° C. and about 460° C. Operating the reactor(s) at these intermediate temperatures may allow one to maximize the amount of desirable products (e.g., aromatic and/or olefin products) produced in a process in which a first reactant (e.g., a solid hydrocarbonaceous material) whose pyrolysis produces a maximum amount of the desired product at a relatively high temperature and a second reactant (e.g., a non-solid reactant comprising, for example, an alcohol such as methanol) whose pyrolysis produces a maximum amount of the desired product at a relatively low temperature are co-fed to the reactor(s). The invention may not be limited to the use of such intermediate temperatures, however, and in other embodiments, lower and/or higher temperatures can be used.

The reactor(s) may also be operated at any suitable pressure. In some embodiments, the reactor may be operated at pressures in the range from about 100 to about 600 kPa (about 1-6 atom), or in the range from about 100 to about 400 kPa (about 1-4 atm), or in the range from about 100 to about 200 kPa (about 1-2 atom). In some embodiments, the reactor may be operated at a pressure below about 600 kPa (about 6 atm), or below about 400 kPa (about 4 atm), or below about 200 kPa (below about 2 atm). In some embodiments, the reactor may be operated at a pressure of at least about 100 kPa (about 1 atm), at least about 200 kPa (about 2 atm), at least about 300 kPa (about 3 atm), or at least about 400 kPa (about 4 atm).

It may be advantageous to heat the feed stream(s) (e.g., the first reactant comprising the solid hydrocarbonaceous material and/or the non-solid second reactant) at a relatively fast rate as it enters the reactor. High heating rates may be advantageous for a number of reasons. For instance, high heating rates may enhance the rate of mass transfer of the reactants from the bulk solid hydrocarbonaceous material and/or the second reactant to the catalytic reactant sites. This may, for example, facilitate introduction of volatile organic compounds formed during the pyrolysis of the solid hydrocarbonaceous material and/or the second reactant into the catalyst before completely thermally decomposing the solid hydrocarbonaceous material and/or the second reactant into generally undesired products (e.g., coke). In addition, high heating rates may reduce the amount of time the reactants are exposed to low temperatures (i.e., temperatures between the temperature of the feed and the desired reaction temperature). Prolonged exposure of the reactants to low temperatures may lead to the formation of undesirable products via undesirable decomposition and/or reaction pathways. Examples of suitable heating rates for heating the feed stream(s) (e.g., including the first reactant comprising solid hydrocarbonaceous material and/or the non-solid second reactant) upon entering the reactor include, for example, greater than about 50° C./s, greater than about 100° C./s, greater than about 200° C./s, greater than about 300° C./s, greater than about 400° C./s, greater than about 500° C./s, greater than about 600° C./s, greater than about 700° C./s, greater than about 800° C./s, greater than about 900° C./s, greater than about 1000° C./s, or greater. In some cases, the first reactant and/or the feed stream may be heated at a heating rate of between about 500° C./s and about 1000° C./s. In some embodiments, the heating rate for heating the feed stream (e.g., containing the first reactant comprising solid hydrocarbonaceous material and/or the non-solid second reactant) upon entering the reactor may be between about 50° C./s and about 1000° C./s, or between about 50° C./s and about 400° C./s. The invention may not limited to the use of such heating rates, however, and in other embodiments, lower and/or higher heating rates can be used.

In some embodiments, the mass-normalized space velocity of the solid hydrocarbonaceous material may be selected to selectively produce a desired array of fluid hydrocarbon products. As used herein, the term “mass-normalized space velocity” of a component is defined as the mass flow rate of the component into the reactor (e.g., as measured in g/hr) divided by the mass of catalyst in the reactor (e.g., as measured in g) and has units of inverse time. For example, the mass-normalized space velocity of solid hydrocarbonaceous material fed to the reactor is calculated as the mass flow rate of the solid hydrocarbonaceous material into the reactor divided by the mass of catalyst in the reactor. The mass-normalized space velocity of a component (e.g., the solid hydrocarbonaceous material) in a reactor may be calculated using different methods depending upon the type of reactor being used. For example, in systems employing batch or semi-batch reactors, wherein the solid hydrocarbonaceous material is not fed continuously to the reactor, the solid hydrocarbonaceous material does not have a mass-normalized space velocity. For systems in which catalyst is fed to and/or extracted from the reactor during reaction (e.g., circulating fluidized bed reactors), the mass-normalized space velocity may be determined by calculating the average amount of catalyst within the volume of the reactor over a period of operation (e.g., steady-state operation).

In some instances, the mass-normalized space velocity of the solid hydrocarbonaceous material fed to the may be less than about 0.9 hour⁻¹, less than about 0.5 hour⁻¹, between about 0.01 hour⁻¹ and about 0.9 hour⁻¹, between about 0.01 hour⁻¹ and about 0.5 hour⁻¹, between about 0.1 hour⁻¹ and about 0.9 hour⁻¹, or between about 0.1 hour⁻¹ and about 0.5 hour⁻¹. The invention may not be limited to the use of such mass-normalized space velocities, however, and in other embodiments, lower and/or higher mass-normalized space velocities can be used.

In certain cases where fluidized bed reactors are used, the feed material (e.g., a solid hydrocarbonaceous material) in the reactor may be fluidized by flowing a fluid stream through the reactor. In the exemplary embodiment of FIG. 1A, a fluid stream 44 is used to fluidize the feed material in reactor 20. Fluid may be supplied to the fluid stream from a fluid source 24 and/or from the product streams of the reactor via a compressor 26 (which will be described in more detail below). As used herein, the term “fluid” means a material generally in a liquid, supercritical, or gaseous state. Fluids, however, may also contain solids such as, for example, suspended or colloidal particles. In some embodiments, it may be advantageous to control the residence time of the fluidization fluid in the reactor. The residence time of the fluidization fluid may be defined as the volume of the reactor divided by the volumetric flow rate of the fluidization fluid. In some cases, the residence time of the fluidization fluid may be at least about 0.2 second, at least about 0.5 second, at least about 1 second, at least about 3 seconds, at least about 6 seconds, at least about 12 seconds, at least about 24 seconds, or at least about 48 seconds. In some cases, the residence time of the fluidization fluid may be from about 0.2 seconds to about 48 seconds, from about 0.5 seconds to about 48 seconds, from about 1 second to about 48 seconds, from about 3 seconds to about 48 seconds, from about 6 seconds to about 48 seconds, from about 12 seconds to about 48 seconds, or from about 24 seconds to about 48 seconds.

Suitable fluidization fluids that may be used in this invention include, for example, inert gases (e.g., helium, argon, neon, etc.), hydrogen, nitrogen, carbon monoxide, and carbon dioxide, among others.

As shown in the illustrative embodiment of FIG. 1A, the products (e.g., fluid hydrocarbon products) formed during the reaction of the reactants (e.g., the solid hydrocarbonaceous material and the second, non-solid reactant) exit the reactor via a product stream 30. In addition to the reaction products, the product stream may, in some cases, comprise unreacted reactant(s), fluidization fluid, and/or catalyst. In one set of embodiments, the desired reaction product(s) (e.g., liquid aromatic hydrocarbons, olefin hydrocarbons, gaseous products, etc.) may be recovered from an effluent stream of the reactor.

As shown in the illustrative embodiment of FIG. 1A, product stream 30 may be fed to an optional solids separator 32. The solids separator may be used, in some cases, to separate the reaction products from catalyst (e.g., at least partially deactivated catalyst) present in the product stream. In addition, the solids separator may be used, in some instances, to remove coke and/or ash from the catalyst. In some embodiments, the solids separator may comprise optional purge stream 33, which may be used to purge coke, ash, and/or catalyst from the solids separator.

The equipment required to achieve the solids separation and/or decoking steps can be readily designed by one of ordinary skill in the art. For example, in one set of the embodiments, the solids separator may comprise a vessel comprising a mesh material that defines a retaining portion and a permeate portion of the vessel. The mesh may serve to retain the catalyst within the retaining portion while allowing the reaction product to pass to the permeate portion. The catalyst may exit the solids separator through a port on the retaining side of the mesh while the reaction product may exit a port on the permeate side of the mesh. Other examples of solids separators and/or decokers are described in more detail in Kirk-Othmer Encyclopedia of Chemical Technology (Online), Vol. 11, Hoboken, N.J.: Wiley-Interscience, c2001—, pages 700-734; and C. D. Cooper and F. C. Alley. Air Pollution Control, A Design Approach. Second Ed. Prospect Heights, Ill.: Waveland Press, Inc. c1994, pages 127-149, incorporated herein by reference.

The solids separator may be operated at any suitable temperature. In some embodiments, the solids separator may be operated at elevated temperatures. For certain reactions, the use of elevated temperatures in the solids separator can allow for additional reforming and/or reaction of the compounds from the reactor. This may allow for the increased formation of desirable products. Not wishing to be bound by any theory, elevated temperatures in the solids separator may provide enough energy to drive endothermic reforming reactions. The solids separator may be operated at a temperature of, for example, between about 25° C. and about 200° C., between about 200° C. and about 500° C., between about 500° C. and about 600° C., or between about 600° C. and about 800° C. In some cases, the solids separator may be operated at temperatures of at least about 500° C., at least about 600° C., at least 700° C., at least 800° C., or higher.

In some cases, it may be beneficial to control the residence time of the catalyst in the solids separator. The residence time of the catalyst in the solids separator is defined as the volume of the solids separator divided by the volumetric flow rate of the catalyst through the solids separator. In some cases, relatively long residence times of the catalyst in the solids separator may be desired in order to facilitate the removal of sufficient amounts of ash, coke, and/or other undesirable products from the catalyst. In addition, by employing relatively long residence times of the catalyst in the solids separator, the pyrolysis products may be further reacted to produce desirable products. In some embodiments, the residence time and temperature in the solids separator are together selected such that a desired product stream is produced. In some embodiments, the residence time of the catalyst in the solids separator may be at least about 1 second, at least about 5 seconds, at least about 10 seconds, at least about 30 seconds, at least about 60 seconds, at least about 120 seconds, at least about 240 seconds, at least about 300 seconds, at least about 600 seconds, or at least about 1200 seconds. Methods for controlling the residence time of the catalyst in the solids separator are known by those skilled in the art. For example, in some cases, the interior wall of the solids separator may comprise baffles that serve to restrict the flow of catalyst through the solids separator and/or increase the path length of fluid flow in the solids separator. Additionally or alternatively, the residence time of the catalyst in the solids separator may be controlled by controlling the flow rate of the catalyst through the solids separator (e.g., by controlling the flow rate of the fluidizing fluid through the reactor).

The solids separator may have any suitable size. For example, the solids separator may have a volume between about 0.1-1 L, 1-50 L, 50-100 L, 100-250 L, 250-500 L, 500-1000 L, 1000-5000 L, 5000-10,000 L, or 10,000-50,000 L. In some instances, the solids separator may have a volume greater than about 1 L, or in other instances, greater than about 10 L, 50 L, 100 L, 250 L, 500 L, 1,000 L, or 10,000 L. Solids separator volumes greater than 50,000 L are also possible. The solids separator may be cylindrical, spherical, or any other shape and may be circulating or non-circulating. In some embodiments, the solids separator may comprise a vessel or other unit operation similar to that used for one or more of the reactor(s) used in the process. The flow of the catalyst in the solids separator may comprise any suitable geometry. For example, the flow path may be substantially straight. In some cases, the solids separator may comprise a flow channel with a serpentine, meandering, helical, or any other suitable shape. The ratio of the length of the flow path of the solids separator (or, in certain embodiments, the path length of the catalyst through the solids separator) to the average diameter of the solids separator channel may comprise any suitable ratio. In some cases, the ratio may be at least about 2:1, at least 5:1, at least 10:1, at least 50:1, at least 100:1, or greater.

As previously mentioned, the solids separator may not be required in all embodiments. For example, for situations in which catalytic fixed bed reactors are employed, the catalyst may be retained within the reactor, and the reaction products may exit the reactor substantially free of catalyst, thus negating the need for a separate separation step.

In the set of embodiments illustrated in FIG. 1A, separated catalyst may exit the solids separator via stream 34. In some embodiments a portion of the separated catalyst may be returned to the reactor via a return pipe, not shown in FIG. 1. In some cases, the catalyst exiting the separator may be at least partially deactivated. The separated catalyst may be fed, in some embodiments, to a regenerator 36 in which any catalyst that was at least partially deactivated may be re-activated. In some embodiments, the regenerator may comprise optional purge stream 37, which may be used to purge coke, ash, and/or catalyst from the regenerator. Methods for activating catalyst are well-known to those skilled in the art, for example, as described in Kirk-Othmer Encyclopedia of Chemical Technology (Online), Vol. 5, Hoboken, N.J.: Wiley-Interscience, c2001—, pages 255-322, incorporated herein by reference.

In some embodiments, a portion of the catalyst may be removed from the reactor through a catalyst exit port (not shown in FIG. 1.). The catalyst removed from the reactor may be partially deactivated and may be passed via a conduit into regenerator 36, or into a separate regenerator (not shown in FIG. 1). Removed catalyst that has been regenerated may be returned to the reactor via stream 47, or may be returned to the reactor separately from the fluidization gas via a separate stream (not shown in FIG. 1.).

In one set of embodiments, an oxidizing agent may be fed to the regenerator via a stream 38, e.g., as shown in FIG. 1A. The oxidizing agent may originate from any source including, for example, a tank of oxygen, atmospheric air, steam, among others. In the regenerator, the catalyst is re-activated by reacting the catalyst with the oxidizing agent. In some cases, the deactivated catalyst may comprise residual carbon and/or coke, which may be removed via reaction with the oxidizing agent in the regenerator. The regenerator in FIG. 1A comprises a vent stream 40 which may include regeneration reaction products, residual oxidizing agent, etc.

The regenerator may be of any suitable size mentioned above in connection with the reactor or the solids separator. In addition, the regenerator may be operated at elevated temperatures in some cases (e.g., at least about 300° C., 400° C., 500° C., 600° C., 700° C., 800° C., or higher). The residence time of the catalyst in the regenerator may also be controlled using methods known by those skilled in the art, including those outlined above. In some instances, the mass flow rate of the catalyst through the regenerator will be coupled to the flow rate(s) in the reactor and/or solids separator in order to preserve the mass balance in the system.

As shown in the illustrative embodiment of FIG. 1A, the regenerated catalyst may exit the regenerator via stream 42. The regenerated catalyst may be recycled back to the reactor via recycle stream 47. In some cases, catalyst may be lost from the system or removed intentionally during operation. In some such and other cases, additional “makeup” catalyst may be added to the system via a makeup stream 46. As shown illustratively in FIG. 1A, the regenerated and makeup catalyst may be fed to the reactor with the fluidization fluid via recycle stream 47, although in other embodiments, the catalyst and fluidization fluid may be fed to the reactor via separate streams.

Referring back to solids separator 32 in FIG. 1A, the reaction products (e.g., fluid hydrocarbon products) can exit the solids separator via stream 48. In some cases, a fraction of stream 48 may be purged via purge stream 60. The contents of the purge stream may be fed to a combustor or a water-gas shift reactor, for example, to recuperate energy that would otherwise be lost from the system. In some cases, the reaction products in stream 48 may be fed to an optional condenser 50. The condenser may comprise a heat exchanger which condenses at least a portion of the reaction product from a gaseous to a liquid state. The condenser may be used to separate the reaction products into gaseous, liquid, and solid fractions. The operation of condensers is well known to those skilled in the art. Examples of condensers are described in more detail in Perry's Chemical Engineers' Handbook, Section 11: “Heat Transfer Equipment.” 8th ed. New York: McGraw-Hill, c2008, incorporated herein by reference.

The condenser may also, in some embodiments, make use of pressure change to condense portions of the product stream. In FIG. 1A, stream 54 may comprise the liquid fraction of the reaction products (e.g., water, aromatic compounds, olefin compounds, etc.), and stream 74 may comprise the gaseous fraction of the reaction products (e.g., CO, CO₂, H₂, etc.). In some embodiments, the gaseous fraction may be fed to a vapor recovery system 70. The vapor recovery system may be used, for example, to recover any desirable vapors within stream 74 and transport them via stream 72. In addition, stream 76 may be used to transport CO, CO₂, and/or other non-recoverable gases from the vapor recovery system. It should be noted that, in some embodiments, the optional vapor recovery system may be placed in other locations. For example, in some embodiments, a vapor recovery system may be positioned downstream of purge stream 54. One skilled in the art can select an appropriate placement for a vapor recovery system.

Other products (e.g., excess gas) may be transported to optional compressor 26 via stream 56, where they may be compressed and used as fluidization gas in the reactor (stream 22) and/or where they may assist in transporting the hydrocarbonaceous material to the reactor (streams 58) or may be used to transport catalyst to the reactor (not shown), or may be used to transport additional non-solid feeds to the reactor. In some instances, the liquid fraction may be further processed, for example, to separate the water phase from the organic phase, to separate individual compounds, etc.

It should be understood that, while the set of embodiments described by FIG. 1A includes a reactor, solids separator, regenerator, condenser, etc., not all embodiments will involve the use of these elements. For example, in some embodiments, the feed stream(s) may be fed to a catalytic fixed bed reactor, reacted, and the reaction products may be collected directly from the reactor and cooled without the use of a dedicated condenser. In some instances, while a dryer, grinding system, solids separator, regenerator, condenser, and/or compressor may be used as part of the process, one or more of these elements may comprise separate units not fluidically and/or integrally connected to the reactor. In other embodiments one or more of the dryer, grinding system, solids separator, regenerator, condenser, and/or compressor may be absent. In some embodiments, the desired reaction product(s) (e.g., liquid aromatic hydrocarbons, olefin hydrocarbons, gaseous products, etc.) may be recovered at any point in the production process (e.g., after passage through the reactor, after separation, after condensation, etc.).

In some embodiments, a process of the invention may involve the use of more than one reactor. For instance, multiple reactors may be connected in fluid communication with each other, for example, to operate in series and/or in parallel, as shown in the exemplary embodiment of FIG. 1B. In some embodiments, the process may comprise providing a first reactant comprising a solid hydrocarbonaceous material and a second, non-solid reactant in a first reactor and pyrolyzing, within the first reactor, at least a portion of the solid hydrocarbonaceous material under reaction conditions sufficient to produce one or more pyrolysis products. In some embodiments, a catalyst may be provided to the first reactor, and at least a portion of the one or more pyrolysis products and/or at least a portion of the second, non-solid reactant in the first reactor are catalytically reacted using the catalyst under reaction conditions sufficient to produce one or more fluid hydrocarbon products. In some embodiments, the process further comprises catalytically reacting at least a portion of the one or more pyrolysis products and/or the second, non-solid reactant in a second reactor using a catalyst under reaction conditions sufficient to produce one or more fluid hydrocarbon products. In some cases, after catalytically reacting at least a portion of the one or more pyrolysis products and/or the second, non-solid reactant in the second reactor, the process may comprise a step of further reacting within the second reactor at least a portion of the one or more fluid hydrocarbon products from the first reactor (and, optionally, at least a portion of the second, non-solid reactant) to produce one or more other hydrocarbon products.

In FIG. 1B, the reaction product from reactor 20 is transported to a second reactor 20′. Those skilled in the art are familiar with the use of multiple-reactor systems for the pyrolysis of organic material to produce organic products and such systems are known in the art. While FIG. 1B illustrates a set of embodiments in which the reactors are in fluid communication with each other, in some instances, the two reactors may not be in fluid communication. For example, a first reactor may be used to produce a first reaction product which may be transported to a separate facility for reaction in a second reactor. In some instances, a composition comprising a solid hydrocarbonaceous material (with or without a catalyst) may be heated in a first reactor, and at least a portion of the solid hydrocarbonaceous material may be pyrolyzed to produce a pyrolysis product (and optionally at least partially deactivated catalyst). The first pyrolysis product may be in the form of a liquid and/or a gas. The composition comprising the first pyrolysis product may then be heated in a second reactor, which may or may not be in fluid communication with the first reactor. After the heating step in the second reactor, a second pyrolysis product from the second reactor may be collected. The second pyrolysis product may be in the form of a liquid and/or a gas. In some cases, the composition comprising hydrocarbonaceous material that is fed into the first reactor may comprise, for example, a mixture of a solid hydrocarbonaceous material and a solid catalyst. The first pyrolysis product produced from the first reactor may be different in chemical composition, amount, state (e.g., a fluid vs. a gas) than the second pyrolysis product. For example, the first pyrolysis product may substantially include a liquid, while the second pyrolysis product may substantially include a gas. In another example, the first pyrolysis product includes a fluid product (e.g., a bio-oil, sugar), and the second pyrolysis product comprises a relatively higher amount of aromatics than the first pyrolysis product. In some instances, the first pyrolysis product includes a fluid product (e.g., including aromatic compounds), and the second pyrolysis product comprises a relatively higher amount of olefins than the first pyrolysis product. In yet another example, the first pyrolysis product includes a fluid product (e.g., a bio-oil, sugar), and the second pyrolysis product comprises a relatively higher amount of oxygenated aromatic compounds than the first pyrolysis product. In any of these embodiments, a second, non-solid reactant can be fed to the first and/or second reactors, and optionally catalytically reacted with a solid hydrocarbonaceous material and/or a pyrolysis product.

One or more of the reactors in a multiple reactor configuration may comprise a fluidized bed reactor (e.g., a circulating fluidized bed reactor, a turbulent fluidized bed reactor, etc.) or, in other instances, any other type of reactor (e.g., any of the reactors mentioned above). For example, in one set of embodiments, the first reactor comprises a circulating fluidized bed reactor or a turbulent fluidized bed reactor, and the second reactor comprises a circulating fluidized bed reactor or a turbulent fluidized bed reactor in fluid communication with the first reactor. In addition, the multiple reactor configuration may include any of the additional processing steps and/or equipment mentioned herein (e.g., a solids separator, a regenerator, a condenser, etc.). The reactors and/or additional processing equipment may be operated using any of the processing parameters (e.g., temperatures, residence times, etc.) mentioned herein.

Catalyst components useful in the context of this invention can be selected from any catalyst known in the art, or as would be understood by those skilled in the art made aware of this invention. Functionally, catalysts may be limited only by the capability of any such material to promote and/or effect dehydration, dehydrogenation, isomerization, hydrogen transfer, aromatization, decarbonylation, decarboxylation, aldol condensation and/or any other reaction or process associated with or related to the pyrolysis of a hydrocarbonaceous material. Catalyst components can be considered acidic, neutral or basic, as would be understood by those skilled in the art.

The catalyst particles described herein can comprise polycrystalline solids (e.g., polycrystalline particles) in some cases. The catalyst particles can also comprise single crystals, in some embodiments. In certain cases, the particles may be distinct and separate physical objects that are stand-alone. In other cases, the particles may, at least at certain points in their preparation and/or use, comprise an agglomerate of a plurality of individual particles in intimate contact with each other.

A catalyst used in embodiments described herein (e.g., in the feed stream, in the reactor, etc.) may be of any suitable size. In some cases, it may be advantageous to use catalysts comprising relatively small catalyst particles, which may, as mentioned previously, in certain embodiments, be in the form of larger catalyst objects that may be comprised of a plurality of agglomerated catalyst particles. In some embodiments, for example, the use of small catalyst particles may increase the extent to which the hydrocarbonaceous material may contact the surface sites of the catalyst due to, for example, increased external catalytic surface area and decreased diffusion distances through the catalyst. In some cases, catalyst size and/or catalyst particle size may be chosen based at least in part on, for example, the type of fluid flow desired and the catalyst lifetime. Suitable catalysts that may be used with or without modification are available commercially.

In some embodiments, the average diameter (as measured by conventional sieve analysis) of catalyst objects, which may in certain instances each comprise a single catalyst particle or in other instances comprise an agglomerate of a plurality of particles, may be less than about 5 mm, less than about 2 mm, less than about 1 mm, less than about 500 microns, less than about 60 mesh (250 microns), less than about 100 mesh (149 microns), less than about 140 mesh (105 microns), less than about 170 mesh (88 microns), less than about 200 mesh (74 microns), less than about 270 mesh (53 microns), or less than about 400 mesh (37 microns), or smaller.

In some embodiments, the catalyst objects can be or be formed of particles having a maximum cross-sectional dimension of less than about 5 microns, less than about 1 micron, less than about 500 nm, less than about 100 nm, between about 100 nm and about 5 microns, between about 500 nm and about 5 microns, between about 100 nm and about 1 micron, or between about 500 nm and about 1 micron. As noted previously, in certain cases, catalyst particles having the dimensions within the ranges noted immediately above may be agglomerated to form discrete catalyst objects having dimensions within the ranges noted in the previous paragraph. As used here, the “maximum cross-sectional dimension” of a particle refers to the largest dimension between two boundaries of a particle. One of ordinary skill in the art would be capable of measuring the maximum cross-sectional dimension of a particle by, for example, analyzing a scanning electron micrograph (SEM) of a catalyst preparation. In embodiments comprising agglomerated particles, the particles should be considered separately when determining the maximum cross-sectional dimensions. In such a case, the measurement would be performed by establishing imaginary boundaries between each of the agglomerated particles, and measuring the maximum cross-sectional dimension of the hypothetical, individuated particles that result from establishing such boundaries. In some embodiments, a relatively large number of the particles within a catalyst have maximum cross-sectional dimensions that lie within a given range. For example, in some embodiments, at least about 50%, at least about 75%, at least about 90%, at least about 95%, or at least about 99% of the particles within a catalyst have maximum cross-sectional dimensions of less than about 5 microns, less than about 1 micron, less than about 500 nm, less than about 100 nm, between about 100 nm and about 5 microns, between about 500 nm and about 5 microns, between about 100 nm and about 1 micron, or between about 500 nm and about 1 micron.

A relatively large percentage of the volume of the catalyst can be occupied by particles with maximum cross-sectional dimensions within a specific range, in some cases. For example, in some embodiments, at least about 50%, at least about 75%, at least about 90%, at least about 95%, or at least about 99% of the sum of the volumes of all the catalyst used is occupied by particles having maximum cross-sectional dimensions of less than about 5 microns, less than about 1 micron, less than about 500 nm, less than about 100 nm, between about 100 nm and about 5 microns, between about 500 nm and about 5 microns, between about 100 nm and about 1 micron, or between about 500 nm and about 1 micron.

In some embodiments, the particles within a catalyst may be substantially the same size. For example, the catalyst can comprise particles with a distribution of dimensions such that the standard deviation of the maximum cross-sectional dimensions of the particles is no more than about 50%, no more than about 25%, no more than about 10%, no more than about 5%, no more than about 2%, or no more than about 1% of the average maximum cross-sectional dimensions of the particles. Standard deviation (lower-case sigma) is given its normal meaning in the art, and may be calculated as:

$$σ = \sqrt{\frac{{{\sum }_{i =}^{n} {D}_{i} - {D}_{avg} )}^{2}}{n - \text{?}}} \\ \text{?} \text{indicates text missing or illegible when filed} \qquad [ 2 ]$$

wherein Dᵢ is the maximum cross-sectional dimension of particle i, D_{avg} is the average of the maximum cross-sectional dimensions of all the particles, and n is the number of particles within the catalyst. The percentage comparisons between the standard deviation and the average maximum cross-sectional dimensions of the particles outlined above can be obtained by dividing the standard deviation by the average and multiplying by 100%.

Using catalysts including particles within a chosen size distribution indicated above can lead to an increase in the yield and/or selectivity of aromatic compounds produced by the reaction of the hydrocarbonaceous material. For example, in some cases, using catalysts containing particles with a desired size range (e.g., any of the size distributions outlined above) can result in an increase in the amount of aromatic compounds in the reaction product of at least about 5%, at least about 10%, or at least about 20%, relative to an amount of aromatic compounds that would be produced using catalysts containing particles with a size distribution outside the desired range (e.g., with a large percentage of particles larger than 1 micron, larger than 5 microns. etc.).

Alternatively, alone or in conjunction with the considerations mentioned above, catalysts can be selected according to pore size (e.g., mesoporous and pore sizes typically associated with zeolites), e.g., average pore sizes of less than about 100 Angstroms, less than about 50 Angstroms, less than about 20 Angstroms, less than about 10 Angstroms, less than about 5 Angstroms, or smaller. In some embodiments, catalysts with average pore sizes of from about 5 Angstroms to about 100 Angstroms may be used. In some embodiments, catalysts with average pore sizes of between about 5.5 Angstroms and about 6.5 Angstroms, or between about 5.9 Angstroms and about 6.3 Angstroms may be used. In some cases, catalysts with average pore sizes of between about 7 Angstroms and about 8 Angstroms, or between about 7.2 Angstroms and about 7.8 Angstroms may be used.

As used herein, the term “pore size” is used to refer to the smallest cross-sectional diameter of a pore. The smallest cross-sectional diameter of a pore may correspond to the smallest cross-sectional dimension (e.g., a cross-sectional diameter) as measured perpendicularly to the length of the pore. In some embodiments, a catalyst with an “average pore size” or a “pore size distribution” of X refers to a catalyst in which the average of the smallest cross-sectional diameters of the pores within the catalyst is about X. It should be understood that “pore size” or “smallest cross sectional diameter” of a pore as used herein refers to the Norman radii adjusted pore size well known to those skilled in the art. Determination of Norman radii adjusted pore size is described, for example, in Cook, M.; Conner, W. C., “How big are the pores of zeolites?” Proceedings of the International Zeolite Conference, 12th, Baltimore, Jul. 5-10, 1998; (1999), 1, pp 409-414, which is incorporated herein by reference in its entirety. As a specific exemplary calculation, the atomic radii for ZSM-5 pores are about 5.5-5.6 Angstroms, as measured by x-ray diffraction. In order to adjust for the repulsive effects between the oxygen atoms in the catalyst, Cook and Conner have shown that the Norman adjusted radii are 0.7 Angstroms larger than the atomic radii (about 6.2-6.3 Angstroms).

One of ordinary skill in the art will understand how to determine the pore size (e.g., minimum pore size, average of minimum pore sizes) in a catalyst. For example, x-ray diffraction (XRD) can be used to determine atomic coordinates. XRD techniques for the determination of pore size are described, for example, in Pecharsky, V. K. et al, “Fundamentals of Powder Diffraction and Structural Characterization of Materials,” Springer Science+Business Media, Inc., New York, 2005, incorporated herein by reference in its entirety. Other techniques that may be useful in determining pore sizes (e.g., zeolite pore sizes) include, for example, helium pycnometry or low pressure argon adsorption techniques. These and other techniques are described in Magee, J. S. et al, “Fluid Catalytic Cracking: Science and Technology,” Elsevier Publishing Company, Jul. 1, 1993, pp. 185-195, which is incorporated herein by reference in its entirety. Pore sizes of mesoporous catalysts may be determined using, for example, nitrogen adsorption techniques, as described in Gregg, S. J. at al, “Adsorption, Surface Area and Porosity,” 2nd Ed., Academic Press Inc., New York, 1982 and Rouquerol, F. et al, “Adsorption by powders and porous materials. Principles, Methodology and Applications,” Academic Press Inc., New York, 1998, both incorporated herein by reference in their entirety. Unless otherwise indicated, pore sizes referred to herein are those determined by x-ray diffraction corrected as described above to reflect their Norman radii adjusted pore sizes.

A screening method may be used to select catalysts with appropriate pore sizes for the conversion of specific pyrolysis product molecules. The screening method may comprise determining the size of pyrolysis product molecules desired to be catalytically reacted (e.g., the molecule kinetic diameters of the pyrolysis product molecules). One of ordinary skill in the art can calculate, for example, the kinetic diameter of a given molecule. The type of catalyst may then be chosen such that the pores of the catalyst (e.g., Norman adjusted minimum radii) are sufficiently large to allow the pyrolysis product molecules to diffuse into and/or react with the catalyst. In some embodiments, the catalysts are chosen such that their pore sizes are sufficiently small to prevent entry and/or reaction of pyrolysis products whose reaction would be undesirable.

The catalyst may be selected from naturally-occurring zeolites, synthetic zeolites and combinations thereof. The catalyst may be a Mordenite Framework Inverted (MFI) type zeolite catalyst, such as a ZSM-5 zeolite catalyst. Catalysts comprising ZSM-5 that may be used with or without modification are available commercially. Optionally, the catalyst may comprise acidic sites. Other types of useful zeolite catalysts may include ferrierite, zeolite Y, zeolite beta, modernite, MCM-22, ZSM-23, ZSM-57, SUZ-4, EU-1, ZSM-11, (S)A1PO-31, SSZ-23, mixtures of two or more thereof, and the like. In other embodiments, non-zeolite catalysts may be used. For example, WOₓ/ZrO₂, aluminum phosphates, etc., may be used.

The catalyst may comprise a metal and/or a metal oxide. Suitable metals and/or oxides may include, for example, nickel, platinum, vanadium, palladium, manganese, cobalt, zinc, copper, chromium, gallium, and/or any of their oxides, among others. The metal and/or metal oxide can be impregnated into the catalyst (e.g., in the interstices of the lattice structure of the catalyst), in some embodiments. The metal or metal oxide can be added to the zeolite by any of a number of techniques known to those skilled in the art, such as, but not limited to, impregnation, ion exchange, vapor deposition, and the like. The zeolite may comprise small amounts of structure stabilizing elements such as phosphorus, lanthanum, rare earths, and the like, typically at levels that are less than about 1% by weight of the zeolite. The catalyst may be conditioned before operation in the process by a wide range of techniques known to those skilled in the art such as, but not limited to, oxidation, calcination, reduction, cyclic oxidation and reduction, steaming, hydrolysis, and the like. The metal and/or metal oxide may be incorporated into the lattice structure of the catalyst. For example, the metal and/or metal oxide may be included during the preparation of the catalyst, and the metal and/or metal oxide may occupy a lattice site of the resulting catalyst (e.g., a zeolite catalyst). As another example, the metal and/or metal oxide may react or otherwise interact with a zeolite such that the metal and/or metal oxide displaces an atom within the lattice structure of the zeolite.

In certain embodiments, a Mordenite Framework Inverted (MFI) zeolite catalyst comprising gallium can be used. For example, a galloaluminosilicate MFI (GaAlMFI) zeolite catalyst can be used. One of ordinary skill in the art would be familiar with GaAlMFI zeolites, which can be thought of as aluminosilicate MFI zeolites in which some of the Al atoms have been replaced with Ga atoms. In some instances, the zeolite catalyst can be in the hydrogen form (e.g., H—GaAlMFI). The galloaluminosilicate MFI catalyst can be a ZSM-5 zeolite catalyst in which some of the aluminum atoms have been replaced with gallium atoms, in some embodiments.

In some instances, the ratio of moles of Si in the galloaluminosilicate zeolite catalyst to the sum of the moles of Ga and Al (i.e., the molar ratio expressed as Si:(Ga+Al)) in the galloaluminosilicate zeolite catalyst can be at least about 15:1, at least about 20:1, at least about 25:1, at least about 35:1, at least about 50:1, at least about 75:1, or higher. In some embodiments, it may be advantageous to employ a catalyst with a ratio of moles of Si in the zeolite to the sum of the moles of Ga and Al of between about 15:1 and about 100:1, from about 15:1 to about 75:1, between about 25:1 and about 80:1, or between about 50:1 and about 75:1. In some instances, the ratio of moles of Si in the galloaluminosilicate zeolite catalyst to the moles of Ga in the galloaluminosilicate zeolite catalyst can be at least about 30:1, at least about 60:1, at least about 120:1, at least about 200:1, between about 30:1 and about 300:1, between about 30:1 and about 200:1, between about 30:1 and about 120:1, or between about 30:1 and about 75:1. The ratio of the moles of Si in the galloaluminosilicate zeolite catalyst to the moles of Al in the galloaluminosilicate zeolite catalyst can be at least about 10:1, at least about 20:1, at least about 30:1, at least about 40:1, at least about 50:1, at least about 75:1, between about 10:1 and about 100:1, between about 10:1 and about 75:1, between about 10:1 and about 50:1, between about 10:1 and about 40:1, or between about 10:1 and about 30:1.

In addition, in some cases, properties of the catalysts (e.g., pore structure, type and/or number of acid sites, etc.) may be chosen to selectively produce a desired product.

It may be desirable, in some embodiments, to employ one or more catalysts to establish a bimodal distribution of pore sizes. In some cases, a single catalyst with a bimodal distribution of pore sizes may be used (e.g., a single catalyst that contains predominantly 5.9-6.3 Angstrom pores and 7-8 Angstrom pores). In other cases, a mixture of two or more catalysts may be employed to establish the bimodal distribution (e.g., a mixture of two catalysts, each catalyst type including a distinct range of average pore sizes). In some embodiments, one of the one or more catalysts comprises a zeolite catalyst and another of the one or more catalysts comprises a non-zeolite catalyst (e.g., a mesoporous catalyst, a metal oxide catalyst, etc.).

For example, in some embodiments at least about 70%, at least about 80%, at least about 90%, at least about 95%, at least about 98%, or at least about 99% of the pores of the one or more catalysts (e.g., a zeolite catalyst, a mesoporous catalyst, etc.) have smallest cross-sectional diameters that lie within a first size distribution or a second size distribution. In some cases, at least about 2%, at least about 5%, or at least about 10% of the pores of the one or more catalysts have smallest cross-sectional diameters that lie within the first size distribution; and at least about 2%, at least about 5%, or at least about 10% of the pores of the one or more catalysts have smallest cross-sectional diameters that lie within the second size distribution. In some cases, the first and second size distributions are selected from the ranges provided above. In certain embodiments, the first and second size distributions are different from each other and do not overlap. An example of a non-overlapping range is 5.9-6.3 Angstroms and 6.9-8.0 Angstroms, and an example of an overlapping range is 5.9-6.3 Angstroms and 6.1-6.5 Angstroms. The first and second size distributions may be selected such that the range are not immediately adjacent one another, an example being pore sizes of 5.9-6.3 Angstroms and 6.9-8.0 Angstroms. An example of a range that is immediately adjacent one another is pore sizes of 5.9-6.3 Angstroms and 6.3-6.7 Angstroms.

As a specific example, in some embodiments one or more catalysts is used to provide a bimodal pore size distribution for the simultaneous production of aromatic and olefin compounds. That is, one pore size distribution may advantageously produce a relatively high amount of aromatic compounds, and the other pore size distribution may advantageously produce a relatively high amount of olefin compounds. In some embodiments, at least about 70%, at least about 80%, at least about 90%, at least about 95%, at least about 98%, or at least about 99% of the pores of the one or more catalysts have smallest cross-sectional diameters between about 5.9 Angstroms and about 6.3 Angstroms or between about 7 Angstroms and about 8 Angstroms. In addition, at least about 2%, at least about 5%, or at least about 10% of the pores of the one or more catalysts have smallest cross-sectional diameters between about 5.9 Angstroms and about 6.3 Angstroms; and at least about 2%, at least about 5%, or at least about 10% of the pores of the one or more catalysts have smallest cross-sectional diameters between about 7 Angstroms and about 8 Angstroms.

In some embodiments, at least about 70%, at least about 80%, at least about 90%, at least about 95%, at least about 98%, or at least about 99% of the pores of the one or more catalysts have smallest cross-sectional diameters between about 5.9 Angstroms and about 6.3 Angstroms or between about 7 Angstroms and about 200 Angstroms. In addition, at least about 2%, at least about 5%, or at least about 10% of the pores of the one or more catalysts have smallest cross-sectional diameters between about 5.9 Angstroms and about 6.3 Angstroms; and at least about 2%, at least about 5%, or at least about 10% of the pores of the one or more catalysts have smallest cross-sectional diameters between about 7 Angstroms and about 200 Angstroms.

In some embodiments, at least about 70%, at least about 80%, at least about 90%, at least about 95%, at least about 98%, or at least about 99% of the pores of the one or more catalysts have smallest cross-sectional diameters that lie within a first distribution and a second distribution, wherein the first distribution is between about 5.9 Angstroms and about 6.3 Angstroms and the second distribution is different from and does not overlap with the first distribution. In some embodiments, the second pore size distribution may be between about 7 Angstroms and about 200 Angstroms, between about 7 Angstroms and about 100 Angstroms, between about 7 Angstroms and about 50 Angstroms, or between about 100 Angstroms and about 200 Angstroms. In some embodiments, the second catalyst may be mesoporous (e.g., have a pore size distribution of between about 2 nm and about 50 nm).

In some embodiments, the bimodal distribution of pore sizes may be beneficial in reacting two or more hydrocarbonaceous feed material components. For example, some embodiments comprise providing a solid hydrocarbonaceous material comprising a first component and a second component in a reactor, wherein the first and second components are different. Examples of compounds that may be used as first or second components include any of the hydrocarbonaceous materials described herein (e.g., sugar cane bagasse, glucose, wood, corn stover, cellulose, hemi-cellulose, lignin, or any others). For example, the first component may comprise one of cellulose, hemi-cellulose and lignin, and the second component comprises one of cellulose, hemicellulose and lignin. The method may further comprise providing first and second catalysts in the reactor. In some embodiments, the first catalyst may have a first pore size distribution and the second catalyst may have a second pore size distribution, wherein the first and second pore size distributions are different and do not overlap. The first pore size distribution may be, for example, between about 5.9 Angstroms and about 6.3 Angstroms. The second pore size distribution may be, for example, between about 7 Angstroms and about 200 Angstroms, between about 7 Angstroms and about 100 Angstroms, between about 7 Angstroms and about 50 Angstroms, or between about 100 Angstroms and about 200 Angstroms. In some cases, the second catalyst may be mesoporous or non-porous.

The first catalyst may be selective for catalytically reacting the first component or a derivative thereof to produce a fluid hydrocarbon product. In addition, the second catalyst may be selective for catalytically reacting the second component or a derivative thereof to produce a fluid hydrocarbon product. The method may further comprise pyrolyzing within the reactor at least a portion of the hydrocarbonaceous material under reaction conditions sufficient to produce one or more pyrolysis products and catalytically reacting at least a portion of the pyrolysis products with the first and second catalysts to produce the one or more hydrocarbon products. In some instances, at least partially deactivated catalyst may also be produced.

In certain embodiments, a method used in combination with embodiments described herein includes increasing the catalyst to hydrocarbonaceous material mass ratio of a composition to increase production of identifiable aromatic compounds. As illustrated herein, representing but one distinction over certain prior catalytic pyrolysis methods, articles and methods described herein can be used to produce discrete, identifiable aromatic, biofuel compounds selected from but not limited to benzene, toluene, propylbenzene, ethylbenzene, methylbenzene, methylethylbenzene, trimethylbenzene, xylenes, indanes, naphthalene, methylnaphthelene, dimethylnaphthalene, ethylnaphthalene, hydrindene, methylhydrindene, and dimethylhydrindene and combinations thereof.

In some embodiments, the reaction chemistry of a catalyst may be affected by adding one or more additional compounds. For example, the addition of a metal to a catalyst may result in a shift in selective formation of specific compounds (e.g., addition of metal to alumina-silicate catalysts may result in the production of more CO). In addition, when the fluidization fluid comprises hydrogen, the amount of coke formed on the catalyst may be decreased.

In some embodiments, the catalyst may comprise both silica and alumina (e.g., a zeolite catalyst). The silica (SiO₂) and alumina (Al₂O₃) in the catalyst may be present in any suitable molar ratio. For example, in some cases, the catalyst in the feed may comprise a silica (SiO₂) to alumina (Al₂O₃) molar ratio of between about 10:1 and about 50:1, between about 20:1 and about 40:1, or between about 25:1 and about 35:1. In some embodiments, the catalyst in the feed may comprise a silica (SiO₂) to alumina (Al₂O₃) molar ratio of at least about 30:1, at least about 40:1, at least about 50:1, at least about 75:1, at least about 100:1, at least about 150:1, or higher.

In some embodiments, catalyst and solid hydrocarbonaceous material may be present in any suitable ratio. For example, the catalyst and solid hydrocarbonaceous material may be present in any suitable mass ratio in cases where the feed composition (e.g., through one or more feed streams comprising catalyst and solid hydrocarbonaceous material or through separate catalyst and solid hydrocarbonaceous material feed streams), comprises catalyst and solid hydrocarbonaceous material (e.g., circulating fluidized bed reactors). As another example, in cases where the reactor is initially loaded with a mixture of catalyst and solid hydrocarbonaceous material (e.g., a batch reactor), the catalyst and solid hydrocarbonaceous material may be present in any suitable mass ratio. In some embodiments involving circulating fluidized bed reactors, the mass ratio of the catalyst to solid hydrocarbonaceous material in the feed stream—i.e., in a composition comprising a solid catalyst and a solid hydrocarbonaceous material provided to a reactor—may be at least about 0.5:1, at least about 1:1, at least about 2:1, at least about 5:1, at least about 10:1, at least about 15:1, at least about 20:1, or higher. In some embodiments involving circulating fluidized bed reactors, the mass ratio of the catalyst to solid hydrocarbonaceous material in the feed stream may be less than about 0.5:1, less than about 1:1, less than about 2:1, less than about 5:1, less than about 10:1, less than about 15:1, or less than about 20:1; or from about 0.5:1 to about 20:1, from about 1:1 to about 20:1, or from about 5:1 to about 20:1. Employing a relatively high catalyst to solid hydrocarbonaceous material mass ratio may facilitate introduction of the volatile organic compounds, formed from the pyrolysis of the feed material, into the catalyst before they thermally decompose to coke. Not wishing to be bound by any theory, this effect may be at least partially due to the presence of a stoichiometric excess of catalyst sites within the reactor.

In another aspect, a process product is described. In one set of embodiments, a product (e.g., a pyrolysis product) comprises a fluid composition comprising a portion of a reaction product of a solid hydrocarbonaceous material. Such products can be isolated for use as specialty chemicals (e.g., used as fuel directly or as high octane fuel additives) or, alternatively, hydrogenated for use as a biofuel. The products can also be further processed to make other useful compounds.

In some embodiments, the articles and methods described herein are configured to selectively produce aromatic compounds, e.g., in a single-stage, or alternatively, a multi-stage pyrolysis apparatus. For example, in some embodiments, the mass yield of the aromatic compounds in the fluid hydrocarbon product may be at least about 18 wt %, at least about 20 wt %, at least about 25 wt %, at least about 30 wt %, at least about 35 wt %, at least about 39 wt %, between about 18 wt % and about 40 wt %, between about 18 wt % and about 35 wt %, between about 20 wt % and about 40 wt %, between about 20 wt % and about 35 wt %, between about 25 wt % and about 40 wt %, between about 25 wt % and about 35 wt %, between about 30 wt % and about 40 wt %, or between about 30 wt % and about 35 wt %. As used herein, the mass yield of aromatic compounds in a given product is calculated as the total weight of the aromatic compounds present in the fluid hydrocarbon product divided by the combined weight of the solid hydrocarbonaceous material and the non-solid reactant used in forming the reaction product, multiplied by 100%. As used herein, the term “aromatic compound” is used to refer to a hydrocarbon compound comprising one or more aromatic groups such as, for example, single aromatic ring systems (e.g., benzyl, phenyl, etc.) and fused polycyclic aromatic ring systems (e.g. naphthyl, 1,2,3,4-tetrahydronaphthyl, etc.). Examples of aromatic compounds include, but are not limited to, benzene, toluene, indane, indene, 2-ethyl toluene, 3-ethyl toluene, 4-ethyl toluene, trimethyl benzene (e.g., 1,3,5-trimethyl benzene, 1,2,4-trimethyl benzene, 1,2,3-trimethyl benzene, etc.), ethylbenzene, methylbenzene, propylbenzene, xylenes (e.g., p-xylene, m-xylene, o-xylene, etc.), naphthalene, methyl-naphthalene (e.g., 1-methyl naphthalene, anthracene, 9.10-dimethylanthracene, pyrene, phenanthrene, dimethyl-naphthalene (e.g., 1,5-dimethylnaphthalene, 1,6-dimethylnaphthalene, 2,5-dimethylnaphthalene, etc.), ethyl-naphthalene, hydrindene, methyl-hydrindene, and dymethyl-hydrindene. Single ring and/or higher ring aromatics may be produced in some embodiments. The aromatic compounds may have carbon numbers from, for example, C₅-C₁₄, C₆-C₈, C₆-C₁₂, C₈-C₁₂, C₁₀-C₁₄.

In some embodiments, the articles and methods described herein are configured to selectively produce olefin compounds, e.g., in a single-stage, or alternatively, a multi-stage pyrolysis apparatus. In some embodiments, the mass yield of olefin compounds in a fluid hydrocarbon product (e.g., liquid and/or gaseous pyrolysis product) is at least about 3 wt %, at least about 7 wt %, at least about 10 wt %, at least about 12.5 wt %, at least about 15 wt %, at least about 20 wt %, or more. As used herein, the mass yield of olefin compounds in a given product is calculated as the total weight of the olefin compounds present in the fluid hydrocarbon product divided by the combined weight of the solid hydrocarbonaceous material and the non-solid reactant used in forming the reaction product, multiplied by 100%. As used herein, the terms “olefin” or “olefin compound” (a.k.a. “alkenes”) are given their ordinary meaning in the art, and are used to refer to any unsaturated hydrocarbon containing one or more pairs of carbon atoms linked by a double bond. Olefins include both cyclic and acyclinc (aliphatic) olefins, in which the double bond is located between carbon atoms forming part of a cyclic (closed-ring) or of an open-chain grouping, respectively. In addition, olefins may include any suitable number of double bonds (e.g., monoolefins, diolefins, triolefins, etc.). Examples of olefin compounds include, but are not limited to, ethene, propene, butene, butadiene, and isoprene, among others. The olefin compounds may have carbon numbers from, for example, C₂-C₄, C₂-C₈, C₄-C₈, or C₂-C₁₂.

Process conditions may be chosen, in some cases, such that aromatic and/or olefin compounds are selectively produced, e.g., in a single-stage, or alternatively, a multi-stage pyrolysis apparatus. For example, in some embodiments, aromatic and/or olefin compounds may be selectively produced when feeds containing hydrogen to carbon effective ratios of between about 0.75 and about 1.5 (or between about 0.9 and about 1.5, between about 1.0 and about 1.4, or between about 1.2 and about 1.3) are employed. In some embodiments, aromatic and/or olefin compounds may be selectively produced when the mass-normalized space velocity of the solid hydrocarbonaceous material fed to the reactor is less than about 0.9 hour⁻¹ (or, in some instances, less than about 0.5 hour⁻¹, between about 0.01 hour⁻¹ and about 0.9 hour⁻¹, between about 0.01 hour⁻¹ and about 0.5 hour⁻¹, between about 0.1 hour⁻¹ and about 0.9 hour⁻¹, or between about 0.1 hour⁻¹ and about 0.5 hour⁻¹). In some instances, aromatic and/or olefin compounds may be selectively produced when the reactor is operated at a temperature of between about 400° C. and about 600° C. (or between about 425° C. and about 500° C., or between about 440° C. and about 460° C.). In addition, certain heating rates (e.g., at least about 50° C./s, or at least about 400° C./s), high catalyst-to-feed mass ratios (e.g., at least about 5:1), and/or high silica to alumina molar ratios in the catalyst (e.g., at least about 30:1) may be used to facilitate selective production of aromatic and/or olefin compounds. Some such and other process conditions may be combined with a particular reactor type, such as a fluidized bed reactor (e.g., a circulating fluidized bed reactor), to selectively produce aromatic and/or olefin compounds.

Furthermore, in some embodiments, the catalyst may be chosen to facilitate selective production of aromatic and/or olefin products. For example, ZSM-5 may, in some cases, preferentially produce relatively higher amounts of aromatic and/or olefin compounds. In some cases, catalysts that include Bronsted acid sites may facilitate selective production aromatic compounds. In addition, catalysts with well-ordered pore structures may facilitate selective production of aromatic compounds. For example, in some embodiments, catalysts with average pore diameters between about 5.9 Angstroms and about 6.3 Angstroms may be particularly useful in producing aromatic compounds. In addition, catalysts with average pore diameters between about 7 Angstroms and about 8 Angstroms may be useful in producing olefins. In some embodiments, a combination of one or more of the above process parameters may be employed to facilitate selective production of aromatic and/or olefin compounds. The ratio of aromatics to olefins produced may be, for example, between about 0.1:1 and about 10:1, between about 0.2:1 and about 5:1, between about 0.5:1 and about 2:1, between about 0.1:1 and about 0.5:1, between about 0.5:1 and about 1:1, between about 1:1 and about 5:1, or between about 5:1 and about 10:1.

In some embodiments, the catalyst to hydrocarbonaceous material mass ratio in the feed is adjusted to produce desirable products and/or favorable yields. As such, the catalyst to hydrocarbonaceous material mass ratio may be, for example, at least about 0.5:1, at least about 1:1, at least about 2:1, at least about 5:1, at least about 10:1, at least about 15:1, at least about 20:1, or higher in some embodiments; or, less than about 0.5:1, less than about 1:1, less than about 2:1, less than about 5:1, less than about 10:1, less than about 15:1, or less than about 20:1 in other embodiments.

In some embodiments, the process product may also comprise a high-octane biofuel composition comprising a pyrolysis product of a solid hydrocarbonaceous material. The pyrolysis product may be made using a single-stage pyrolysis apparatus, or alternatively, a multi-stage pyrolysis apparatus. In some cases, the solid hydrocarbonaceous material may be mixed with a catalyst (e.g., a zeolite catalyst) during the pyrolysis reaction. The composition may include, for example, discrete, identifiable aromatic compounds, with one, more than one, or each such compound characterized by an octane number greater than or equal to about 90, e.g., at least 92, 95, or 98. As distinguishable over some viscous tars and sludges of the prior art, such a biofuel composition can be characterized as soluble in petroleum-derived gasolines, diesel fuels and/or heating fuels. Such compounds can include, but are not limited to, benzene, toluene, ethylbenzene, methylethylbenzene, trimethylbenzene, xylenes, indanes naphthalene, methylnaphthelene, dimethylnaphthalene, ethylnaphthalene, hydrindene, methylhydrindene, and dimethylhydrindene and combinations thereof, the identity and/or relative amounts of which can vary depending upon choice of biomass composition, catalyst type, and/or any of the process parameters described herein.

In some embodiments, the process product may comprise a non-acidic biofuel compatible with existing gasoline and diesel fuel lines.

Furthermore, processes described herein may result in lower coke formation than certain existing methods. For example, in some embodiments, a pyrolysis product can be formed with less than about 30 wt %, less than about 25 wt %, less than about 20 wt %, than about 15 wt %, or less than about 10 wt % of the pyrolysis product being coke. The amount of coke formed is measured as the weight of coke formed in the system divided by the weight of hydrocarbonaceous material used in forming the pyrolysis product.

The following non-limiting examples are intended to illustrate various aspects and features of the invention.

### Example

This example describes the catalytic fast pyrolysis (CFP) of pine wood, alcohols (methanol, 1-propanol, 1-butanol and 2-butanol) and their mixtures with ZSM-5 catalyst in a bubbling fluidized bed reactor to determine whether the overall petrochemical yield can be improved by the addition of non-solid feeds comprising hydrogen. The effect of temperature and weight hourly space velocity (WHSV) on the product carbon yields and selectivities of CFP of pine wood and methanol are studied to determine suitable operation conditions for co-CFP of these feed mixtures. Isotopically-labeled ¹³C methanol is processed with pine wood to identify how methanol and wood are incorporated into the final products. Also, the co-CFP of pine wood with other alcohols (such as 1-propanol, 1-butanol and 2-butanol) is performed to determine if these feeds can be used to enhance the petrochemical yield. This example provides data as to how the yield of petrochemicals can be increased by co-feeding pine wood with reactants (like alcohols) to produce a high H/Ceff ratio in the feed.

Unlabeled (i.e., ¹²C, or naturally occurring) methanol, 1-propanol, 1-butanol and 2-butanol are purchased from Sigma-Aldrich and used as feedstocks without any pretreatment. Isotopically enriched ¹³C methanol (Product ID: CLM-359-5) with 99 atom % ¹³C is bought from Cambridge Isotope Laboratories, Inc. The wood used in this study is eastern pine wood sawdust sourced from a sawmill in Amherst, Mass. (W.D.Cowls, Inc. Land Company). Prior to the experiments, the wood is ground with a high-speed rotary cutting mill and then sieved to yield a particle size of 18-120 mesh (880-120 micrometers). The elemental composition of the wood is 46.2 wt % carbon, 6.0 wt % hydrogen, and 47.3 wt % oxygen (by difference). The approximate analysis result is 4.0 wt % moisture, 74.2 wt % volatile, 21.3 wt % fixed carbon, and 0.5 wt % ash. On the dry basis the approximate molecular formula of the wood is C_{3.8}H_{5.8}O_{2.7}.

The catalyst is a spray-dried 40% ZSM-5 catalyst. The particle size of the catalyst is from 150 to 230 mesh (106-62 micrometers). For a typical run, 30 grams of catalyst are loaded into the fluidized bed reactor. Prior to reactions, the catalyst is calcined in the fluidized bed reactor for 5 hours at 600° C. in 800 ml min⁻¹ flowing air.

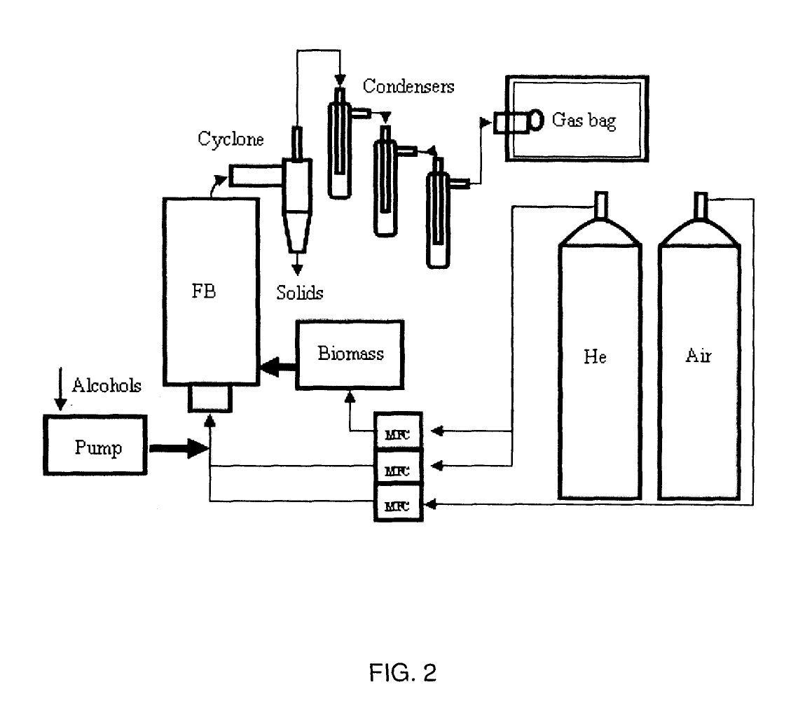

CFP and co-CFP of pine wood and alcohols are conducted in a bubbling fluidized bed reactor system as shown in FIG. 2. The fluidized bed reactor is a 2-inch (5.08 cm) ID 316 stainless steel tube with a freeboard height of 10 inches (25.4 cm). The catalyst bed is supported by a distributor plate made from stacked 316 stainless steel mesh (300 mesh). The pine wood is fed into the side of the reactor (1 inch (2.54 cm) above the distributor plate) by a stainless steel auger from a sealed feed hopper. The hopper is swept with helium at a rate of 200 mL min⁻¹ to maintain an inert environment in the feeding unit. Methanol is fed into the fluidizing gas stream at the inlet of the plenum by a syringe pump. The methanol vaporizes in the gas stream and enters the reactor through the distributor plate as vapor. Ultra high purity helium (99.99%) was used as the fluidizing gas. The gas flow rate is set to 1200 mL min⁻¹ and controlled by a mass flow controller. Both the reactor and the inlet gas stream are heated to reaction temperature before reaction. A cyclone is located at the exit of the reactor to remove and collect entrained solid particles. Following the cyclone, the vapors flow into a condenser train. The first three condensers are operated at 0° C. in an ice bath, and the following four condensers are operated at −55° C. in a dry ice/acetone bath. The non-condensable vapors exiting the condenser train are collected by a gas sampling bag and analyzed by a gas chromatograph/flame ionization detector (GC/FID) and a gas chromatograph/thermal conductivity detector (GC/TCD). The liquids in the condensers are collected using ethanol solvent and analyzed by GC/FID. For the isotopically-labeled feed run, the gas samples and liquids are further analyzed by a gas chromatograph/mass spectrometer (GC/MS). For a typical run, pine wood is fed to the reactor for 30 min. At the end of the run, the reactor is purged with 1200 mL min⁻¹ for another 30 min to strip any remaining product from the catalyst. After reaction, the catalyst is regenerated at 600° C. in 800 ml min⁻¹ flowing air. The effluent gas during regeneration contains CO₂, CO and H₂O, and is passed in series through a copper catalyst, a Drierite™ trap and an Ascarite® trap. The copper catalyst is a copper oxide powder (13% CuO on alumina, Sigma-Aldrich) and is operated at 250° C. to convert CO to CO₂. The coke carbon yield is determined by the mass of CO₂ captured by the Ascarite trap.

### Catalytic Fast Pyrolysis of Pine Wood

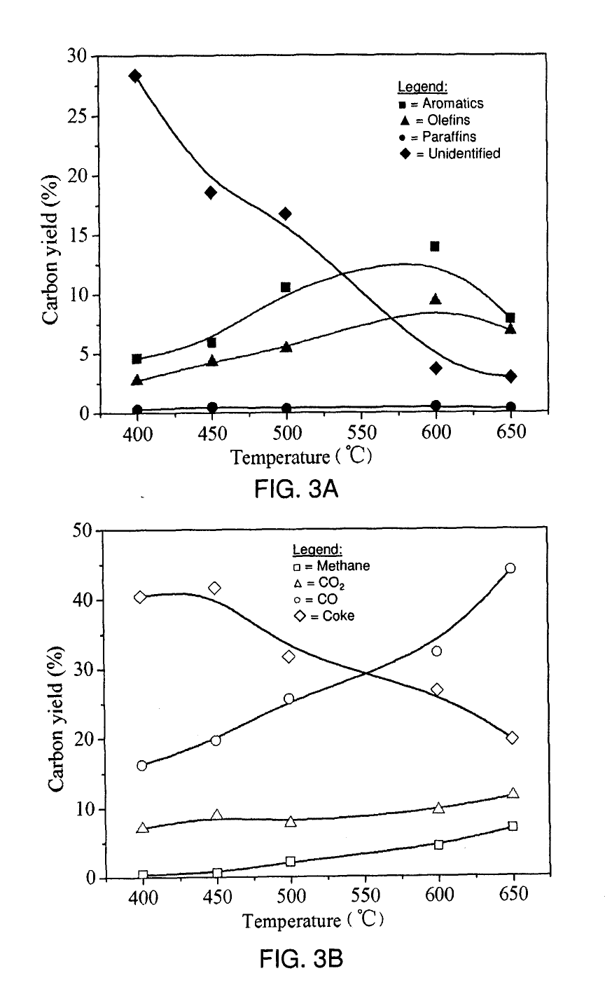

The catalytic fast pyrolysis of pine wood is studied. In one set of experiments, the effects of variation in the reaction temperature on the pyrolysis product yields and selectivities is investigated. For this set of experiments, the reaction conditions are as follows: ZSM-5 catalyst, 0.35 h⁻¹ WHSV, 1200 mL min⁻¹ helium fluidizing flow rate, 30 min reaction time. FIG. 3 and Table 1 show the product carbon yields for CFP of pine wood in the fluidized bed reactor at different temperatures. The aromatic and olefin yields peak at 13.9% and 9.4%, respectively, at 600° C. The maximum total carbon yield of petrochemicals (aromatics+olefins+paraffins) is 23.7%, which occurs at 600° C. The coke and unidentified oxygenates yields decrease with increasing temperature from 40.4% and 28.4%, respectively, at 400° C. to 19.7% and 2.9%, respectively, at 650° C. The CO and methane yields increase with increasing temperature from 16.2% and 0.3%, respectively, at 400° C. to 44.1% and 6.9%, respectively, at 650° C. The detailed product carbon yields and selectivities are listed in Table 1. Aromatic products included benzene, toluene, xylene, and naphthalene. Benzene and naphthalene selectivities increase, while xylene and ethyl benzene selectivities decrease with increasing temperature. Olefin products include ethylene, propylene, butenes, and butadiene. Propylene and butenes selectivities decrease and ethylene selectivity increases with increasing temperature.

TABLE 1
Detailed product yields and selectivities for CFP of pine wood at various temperatures and
WHSV = 0.35 h⁻¹. Aromatic selectivity is defined as the moles of carbon in the product divided by the total
moles aromatic carbon. Olefin selectivity is defined as the moles of carbon in the product divided by the
total moles olefin carbon.

| | Temperature (° C.) | | | | |
|---|---|---|---|---|---|
| Compound | 400 | 450 | 500 | 600 | 650 |
| Overall yields | | | | | |
| Aromatics | 4.6 | 5.9 | 10.5 | 13.9 | 7.8 |
| Olefins | 2.7 | 4.3 | 5.4 | 9.4 | 6.8 |
| Paraffins | 0.3 | 0.5 | 0.3 | 0.5 | 0.3 |
| Petrochemicals | 7.6 | 10.7 | 16.2 | 23.7 | 14.9 |
| Methane | 0.3 | 0.6 | 2.1 | 4.3 | 6.9 |
| CO₂ | 7.1 | 8.9 | 7.7 | 9.5 | 11.6 |
| CO | 16.2 | 19.7 | 25.6 | 32.2 | 44.1 |
| Coke | 40.4 | 41.6 | 31.7 | 26.7 | 19.7 |
| Total balance | 71.7 | 81.4 | 83.3 | 96.4 | 97.1 |
| Unidentified | 28.4 | 18.6 | 16.7 | 3.6 | 2.9 |
| Aromatic selectivity | | | | | |
| Benzene | 13.4 | 10.8 | 15.3 | 20.8 | 38.8 |
| Toluene | 30.9 | 32.2 | 34.7 | 37.1 | 30.7 |
| Ethyl-benzene | 4.9 | 3.4 | 4.5 | 2.3 | 0 |
| p-Xylene and | 29.6 | 33.2 | 24.1 | 16.6 | 8.9 |
| m-Xylene | | | | | |
| o-Xylene | 7.1 | 4.8 | 3.5 | 3.2 | 2.2 |
| Styrene | 0 | 0 | 3.7 | 2.8 | 0 |
| Benzofuran | 3.0 | 4.3 | 1.6 | 1.9 | 0 |
| Indene | 2.4 | 2.6 | 3.3 | 2.6 | 1.8 |
| Phenol | 1.4 | 1.1 | 1.5 | 1.4 | 1.3 |
| Naphthalene | 7.3 | 7.7 | 7.7 | 11.2 | 16.4 |
| Olefin selectivity | | | | | |
| Ethylene | 36.5 | 54.9 | 49.0 | 52.8 | 66.4 |
| Propylene | 48.1 | 36.0 | 37.8 | 36.0 | 26.4 |
| Butenes | 7.2 | 7.2 | 7.0 | 6.0 | 3.8 |
| Butadiene | 8.2 | 1.9 | 6.2 | 5.2 | 3.4 |

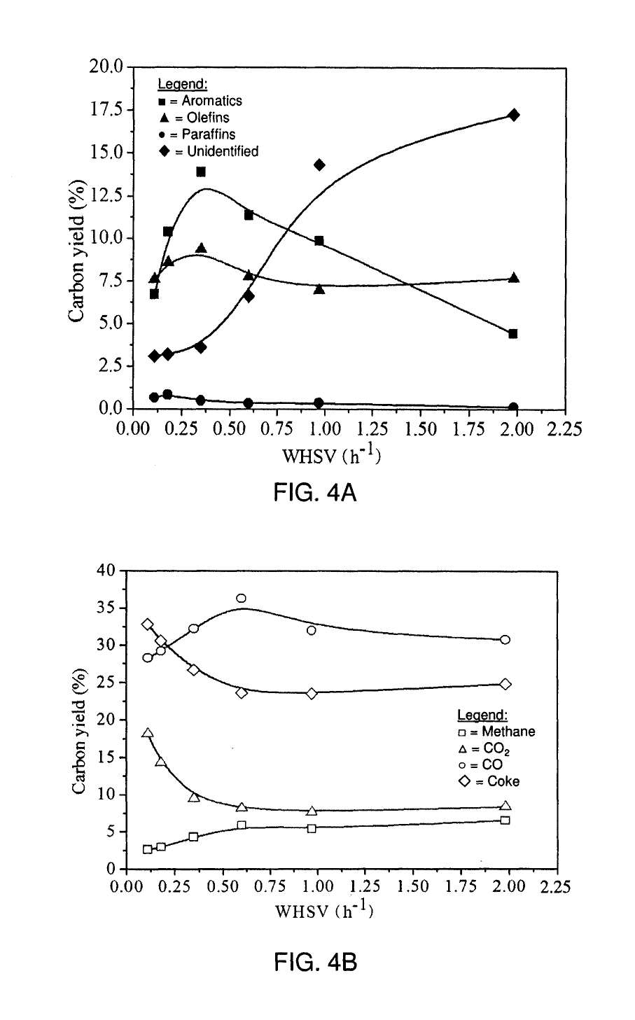

The effects of variations in the weight hourly space velocity on pyrolysis product yields and selectivities are investigated. For this set of experiments, the reaction conditions are as follows: ZSM-5 catalyst, 600° C. temperature, 1200 mL min⁻¹ helium fluidizing flow rate, 30 min reaction time. The product carbon yields for CFP of pine wood at 600° C. as a function of weight hourly space velocity (WHSV) are shown in FIG. 4 and Table 2. WHSV is defined as the mass flow rate of feed divided by the mass of catalyst in the reactor. The aromatic and olefin yields exhibit a maximum at WHSV=0.35 h⁻¹. The unidentified oxygenates yield increases from 3.1% at WHSV=0.11 h⁻¹ to 17.3% at WHSV=1.98 h⁻¹ with increasing WHSV. The methane yield increases from 3.0% to 6.5% with increasing WHSV. The CO yield exhibits a maximum of 36.3% at WHSV=0.60 h⁻¹. The CO₂ and coke yields decrease with increasing WHSV. The xylene and toluene selectivities decrease, whereas the benzene and naphthalene selectivities increase with increasing WHSV. The ethylene and butenes selectivities decrease with the increase of WHSV.

TABLE 2
Detailed product yields and selectivities for CFP of pine wood
at various WHSV values and 600° C. Aromatic selectivity is
defined as the moles of carbon in the product divided by the total
moles aromatic carbon. Olefin selectivity is defined as the moles
of carbon in the product divided by the total moles olefin carbon.

| | WHSV (h⁻¹) | | | | | |
|---|---|---|---|---|---|---|
| Compound | 0.11 | 0.18 | 0.35 | 0.60 | 0.97 | 1.98 |
| Overall yields | | | | | | |
| Aromatics | 6.7 | 10.4 | 13.9 | 11.4 | 9.8 | 4.4 |
| Olefins | 7.6 | 8.6 | 9.4 | 7.8 | 7.0 | 7.7 |
| Parrafins | 0.7 | 0.8 | 0.5 | 0.3 | 0.4 | 0.1 |
| Petrochemicals | 15.0 | 19.8 | 23.7 | 19.5 | 17.2 | 12.3 |
| Methane | 3.0 | 2.9 | 4.3 | 5.9 | 5.3 | 6.5 |
| CO₂ | 18.2 | 14.3 | 9.5 | 8.2 | 7.7 | 8.4 |
| CO | 28.3 | 29.2 | 32.2 | 36.3 | 32.0 | 30.8 |
| Coke | 32.9 | 30.6 | 26.7 | 23.6 | 23.5 | 24.8 |
| Total balance | 96.9 | 96.8 | 96.4 | 93.4 | 85.7 | 82.7 |
| Unidentified | 3.1 | 3.2 | 3.6 | 6.6 | 14.3 | 17.3 |
| Aromatic selectivity | | | | | | |
| Benzene | 25.4 | 21.7 | 20.8 | 24.3 | 29.2 | 36.4 |
| Toluene | 40.1 | 37.2 | 37.1 | 32.0 | 24.8 | 20.9 |
| Ethyl-benzene | 1.7 | 1.8 | 2.3 | 2.0 | 2.6 | 2.0 |
| p-Xylene and | 18.1 | 19.0 | 16.6 | 14.2 | 13.5 | 9.0 |
| m-Xylene | | | | | | |
| o-Xylene | 3.8 | 1.1 | 3.2 | 4.2 | 2.8 | 2.8 |
| Styrene | 0 | 3.8 | 2.8 | 2.0 | 2.7 | 1.6 |
| Benzofuran | 0.3 | 1.6 | 1.9 | 2.2 | 2.8 | 2.9 |
| Indene | 1.4 | 2.3 | 2.6 | 3.5 | 2.6 | 2.3 |
| Phenol | 0.6 | 1.0 | 1.4 | 1.7 | 1.4 | 1.0 |
| Naphthalene | 8.7 | 10.6 | 11.2 | 13.9 | 17.7 | 21.3 |
| Olefin selectivity | | | | | | |
| Ethylene | 55.6 | 49.3 | 52.8 | 61.1 | 53.3 | 49.4 |
| Propylene | 24.9 | 32.3 | 36.0 | 29.5 | 37.0 | 40.1 |
| Butenes | 14.2 | 12.3 | 6.0 | 4.6 | 4.8 | 5.9 |
| Butadience | 5.2 | 6.0 | 5.2 | 4.9 | 4.9 | 4.6 |

### Catalytic Conversion of Methanol

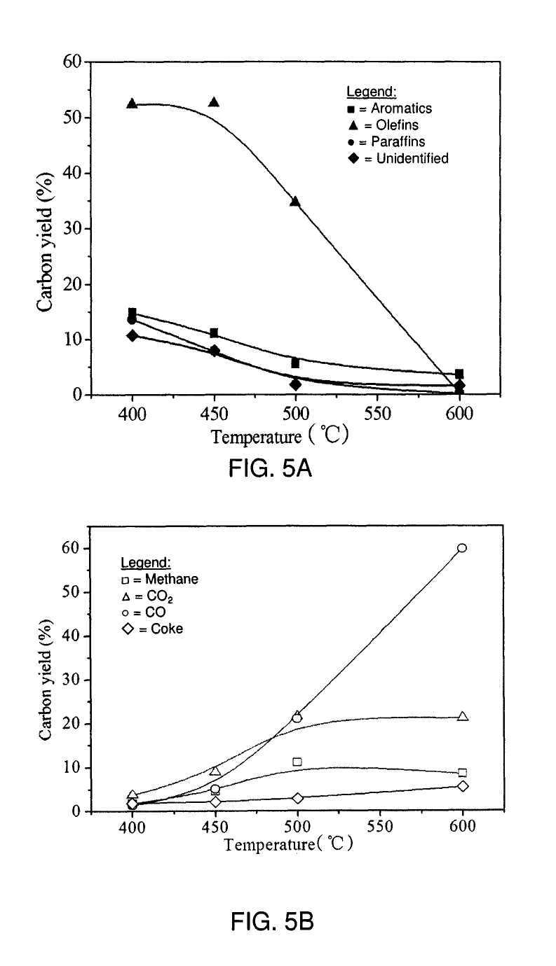

The catalytic conversion of methanol is studied. In one set of experiments, the effects of variations in reaction temperature on product yields and selectivities are investigated. The reaction conditions for this set of experiments are as follows: ZSM-5 catalyst, 0.35 h⁻¹ WHSV, 1200 mL min⁻¹ helium fluidizing flow rate, 30 min reaction time. Product carbon yields of catalytic conversion of methanol at different temperatures with WHSV of 0.35 h⁻¹ are shown in FIG. 5 and Table 3. Temperature has a pronounced effect on the product distribution. The yields of olefins, aromatics, paraffins, and unidentified oxygenates decrease with increasing temperature. The yields of methane, CO₂, CO, and coke increase with increasing temperature. Temperature has the biggest effect on the olefin and CO yields. The olefin yield decreases from 67.1% at 400° C. to 3.7% at 600° C. The CO yield increases from 1.3% at 400° C. to 61.7% at 600° C. The petrochemical yield decreases from 80.9% at 400° C. to 0.1% at 600° C. Table 3 lists the detailed product yields and selectivities. The benzene and toluene selectivities increase and the xylene selectivity decreases with an increase in temperature. At high temperature (600° C.) almost all the methanol is converted into CO, CO₂, methane and coke.

TABLE 3
Detailed product yields and selectivities for catalytic conversion
of methanol at various temperatures and
WHSV = 0.35 h⁻¹. Aromatic sensitivity is defined as the
moles of carbon in the product divided by the total moles
aromatic carbon. Olefin selectivity is defined as the moles of carbon
in the product divided by the total moles olefin carbon.

| | Temperature (° C.) | | | |
|---|---|---|---|---|
| Compound | 400 | 450 | 500 | 600 |
| Overall yields | | | | |
| Aromatics | 14.8 | 11.1 | 5.5 | 3.5 |
| Olefins | 52.3 | 52.6 | 34.6 | 0.2 |
| Parrafins | 13.6 | 7.9 | 1.8 | 0.1 |
| Petrochemicals | 80.7 | 71.5 | 41.9 | 3.8 |
| Methane | 1.8 | 4.5 | 11.0 | 8.4 |
| CO₂ | 3.7 | 8.9 | 21.5 | 21.1 |
| CO | 1.3 | 5.1 | 21.0 | 59.7 |
| Coke | 1.8 | 2.1 | 2.9 | 5.5 |
| Total balance | 89.3 | 92.1 | 98.3 | 98.4 |
| Unidentified | 10.7 | 7.9 | 1.7 | 1.6 |
| Aromatic selectivity | | | | |
| Benzene | 1.8 | 3.3 | 9.6 | 23.7 |
| Toluene | 11.1 | 15.6 | 19.3 | 40.6 |
| Ethyl-benzene | 1.8 | 1.6 | 2.5 | 2.0 |
| p-Xylene and m-Xylene | 57.3 | 56.5 | 43.3 | 20.8 |
| o-Xylene | 9.5 | 9.5 | 7.6 | 1.2 |
| Styrene | 0 | 0 | 4.4 | 4.1 |
| Indene | 18.5 | 13.6 | 11.0 | 2.6 |
| Phenol | 1.5 | 1.0 | 0.8 | 0.5 |
| Naphthalene | 7.7 | 10.8 | 8.6 | 6.7 |
| Olefin selectivity | | | | |
| Ethylene | 14.3 | 18.0 | 20.2 | 0 |
| Propylene | 43.7 | 50.2 | 51.5 | 100 |
| Butenes | 38.3 | 25.9 | 24.9 | 0 |
| Butadiene | 3.7 | 5.9 | 3.4 | 0 |

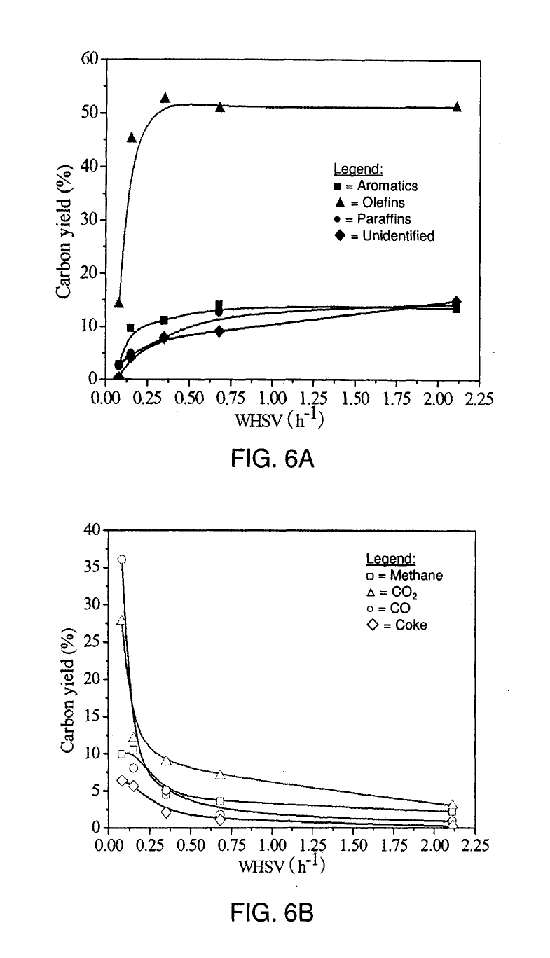

The effects of variations in the weight hourly space velocity on product yields and selectivities is studied. FIG. 6 and Table 4 show product yields as a function of WHSV for catalytic conversion of methanol at 450° C. The petrochemical yields increases rapidly at lower WHSV, while CO, CO₂, methane, and coke yields show the opposite trend. At WHSV above 0.50, little change is observed. The petrochemical yield increases from 19.5% to 59.7% when the WHSV increases from 0.08 h⁻¹ to 0.15 h⁻¹. The petrochemical yield increases to 71.5% at WHSV=0.35 h⁻¹; however, increasing the WHSV further has little effect on the total yield. This indicates that methanol conversion is sensitive at low WHSV. Benzene selectivity decreases, and xylene selectivity increases with increasing WHSV. Ethylene selectivity decreases, and propylene and butenes selectivities increase with an increase in WHSV. These experiments show that the maximum petrochemical yield for methanol conversion occurs at low temperatures and high WHSV.

TABLE 4
Detailed product yields and selectivities for catalytic conversion of methanol at various
WHSV values and 450° C. Aromatic selectivity is defined as the moles of carbon in the
product divided by the total moles aromatic carbon. Olefin selectivity is defined as the
moles of carbon in the product divided by the total moles olefin carbon.

| | WHSV (h⁻¹) | | | | |
|---|---|---|---|---|---|
| Compound | 0.08 | 0.15 | 0.35 | 0.68 | 2.11 |
| Overall yields | | | | | |
| Aromatics | 2.8 | 9.7 | 11.1 | 14.0 | 13.5 |
| Olefins | 14.2 | 45.2 | 52.6 | 50.8 | 51.2 |
| Parrafins | 2.6 | 4.8 | 7.9 | 12.7 | 14.2 |
| Petrochemicals | 19.5 | 59.7 | 71.5 | 77.5 | 78.9 |
| Methane | 9.9 | 10.5 | 4.5 | 3.6 | 2.2 |
| CO₂ | 27.8 | 12.0 | 8.9 | 7.0 | 3.0 |
| CO | 36.0 | 8.1 | 5.1 | 1.8 | 0.9 |
| Coke | 6.4 | 5.7 | 2.1 | 1.1 | 0.3 |
| Total balance | 99.7 | 95.9 | 92.1 | 90.9 | 87.2 |
| Unidentified | 0.3 | 4.1 | 7.9 | 9.1 | 12.8 |
| Aromatic selectivity | | | | | |
| Benzene | 9.2 | 3.8 | 3.3 | 2.9 | 2.2 |
| Toluene | 22.1 | 16.5 | 15.6 | 17.7 | 16.0 |
| Ethyl-benzene | 0 | 1.3 | 1.6 | 2.2 | 2.6 |
| p-Xylene and | 49.5 | 54.9 | 56.5 | 55.7 | 58.8 |
| m-Xylene | | | | | |
| o-Xylene | 13.1 | 10.2 | 9.5 | 9.8 | 9.2 |
| Indene | 6.2 | 13.2 | 13.6 | 11.6 | 11.3 |
| Olefin selectivity | | | | | |
| Ethylene | 35.0 | 17.3 | 18.0 | 14.9 | 12.6 |
| Propylene | 34.1 | 52.7 | 50.2 | 42.6 | 42.3 |
| Butenes | 27.7 | 25.3 | 25.9 | 37.4 | 43.2 |
| Butadiene | 3.2 | 4.7 | 5.9 | 5.1 | 1.9 |

### Co-Catalytic Fast Pyrolysis of Pine Wood and Methanol

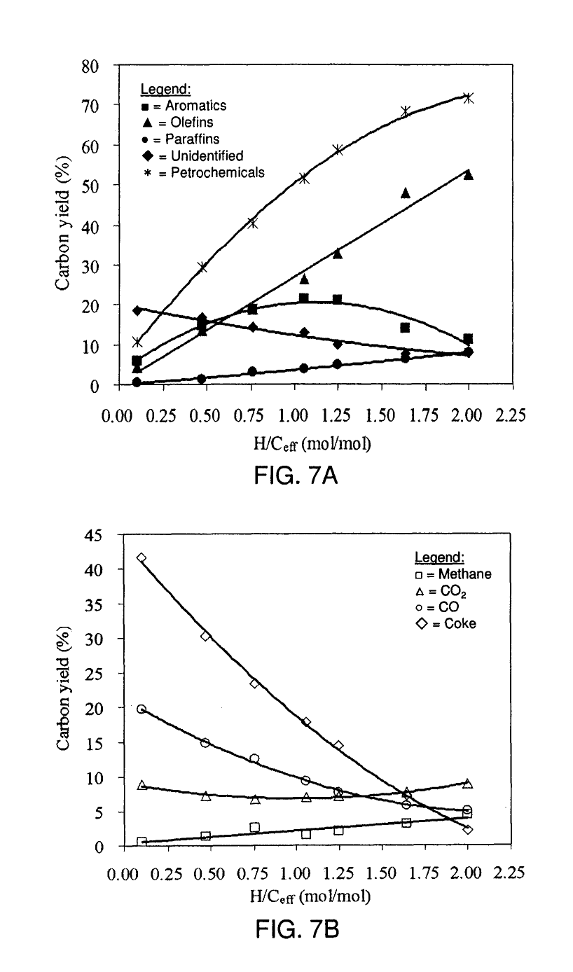

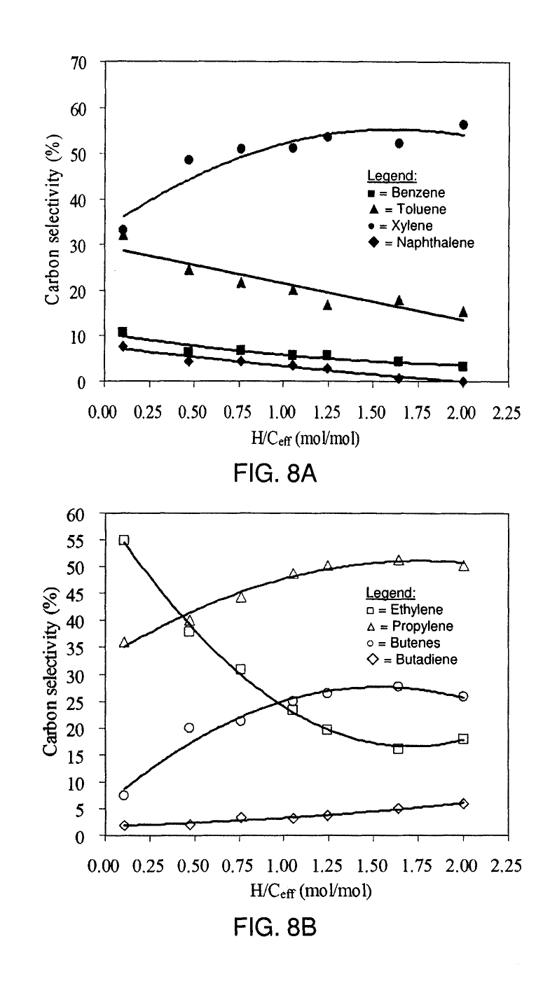

Co-catalytic fast pyrolysis (co-CFP) of pine wood and methanol is carried out by co-feeding the pine wood and methanol to the reactor. In one set of experiments, the effects of varying the hydrogen to carbon effective (H/C_{eff}) ratio at 450° C. are studied. The H/C_{eff} ratio of the feed is adjusted by changing the space velocity ratio of pine wood and methanol. The reactor is operated at temperatures of 450° C. and 500° C. The product yields of co-CFP of pine wood and methanol at 450° C. as a function of H/C_{eff} ratio are shown in FIG. 7 and Table 5. The petrochemical yield increases non-linearly with increasing H/C_{eff} ratio. This curvature indicates that there is a synergistic effect between the feeds since pure addition of the yields would yield a straight line with increasing H/C_{eff} ratio. This synergistic effect of co-feeding is apparent in the aromatic yield. The aromatic yield is 5.9% when only pine wood is fed and 10.9% when only methanol is fed. However, at the intermediate value of H/C_{eff}=1.05, a maximum yield of 21.4% aromatics is realized. This result indicates that the aromatic yield is enhanced by co-feeding methanol and is not purely additive. The unidentified oxygenates yield decreases from 18.6% at H/C_{eff}=0.11 to 7.9% at H/C_{eff}=2. As shown in FIG. 7B, the yields of CO and coke decrease significantly with an increase in the H/C_{eff} ratio as non-linear curves, while that of CO₂ remains constant. FIG. 8 shows the selectivities of benzene, toluene, xylene, and naphthalene in the aromatic products and ethylene, propylene, butenes and butadiene in olefin products for co-CFP of pine wood and methanol at 450° C. As shown in FIG. 8, the selectivities of the more valuable chemicals such as xylene, propylene, butenes, and butadiene increase significantly with an increase in H/C_{eff} ratio, while the selectivities of the less valuable chemicals, such as naphthalene, decrease.

TABLE 5A
Detailed product yields and selectivites for co-CFP of pine wood and methanol
at various H/C_{eff} ratios and 450° C. Aromatic selectivity is defined as the moles
of carbon in the product divided by the total moles aromatic carbon. Olefin
selectivity is defined as the moles of carbon in the product divided by the total
moles olefin carbon. The results in Table 5A are based on experimental data.

| | H/C_{eff} (mol/mol) ratio | | | | | | |
|---|---|---|---|---|---|---|---|
| Compound | 0.11 | 0.47 | 0.76 | 1.05 | 1.25 | 1.64 | 2.00 |
| Pine wood WHSV (h⁻¹) | 0.35 | 0.46 | 0.23 | 0.28 | 0.20 | 0.06 | 0 |
| Methanol WHSV (h⁻¹) | 0 | 0.13 | 0.15 | 0.35 | 0.37 | 0.34 | 0.35 |
| Total WHSV (h⁻¹) | 0.35 | 0.59 | 0.38 | 0.63 | 0.56 | 0.41 | 0.35 |
| Overall yields | | | | | | | |
| Aromatics | 5.9 | 14.8 | 18.8 | 21.4 | 21.1 | 14.0 | 11.1 |
| Olefins | 4.3 | 13.5 | 18.7 | 26.4 | 32.9 | 47.9 | 52.6 |
| Parrafins | 0.5 | 1.2 | 2.9 | 3.7 | 4.7 | 6.3 | 7.9 |
| Petrochemicals | 10.7 | 29.4 | 40.5 | 51.4 | 58.7 | 68.3 | 71.5 |
| Methane | 0.6 | 1.4 | 2.6 | 1.5 | 2.1 | 3.1 | 4.5 |
| CO₂ | 8.9 | 7.3 | 6.8 | 7.1 | 7.2 | 7.8 | 8.9 |
| CO | 19.7 | 14.9 | 12.6 | 9.4 | 7.7 | 5.8 | 5.1 |
| Coke | 41.6 | 30.3 | 23.4 | 17.8 | 14.5 | 7.4 | 2.1 |
| Total balance | 81.4 | 83.3 | 85.8 | 87.2 | 90.2 | 92.3 | 92.1 |
| Unidentified | 18.6 | 16.7 | 14.2 | 12.8 | 9.8 | 7.7 | 7.9 |
| Theoretical petrochemical yield | 64.3 | 71.2 | 76.6 | 82.2 | 85.8 | 93.2 | 100 |
| Aromatic selectivity | | | | | | | |
| Benzene | 10.8 | 6.5 | 6.7 | 5.8 | 5.8 | 4.5 | 3.3 |
| Toluene | 32.2 | 24.6 | 21.8 | 20.2 | 16.9 | 18.0 | 15.6 |
| Ethyl-benzene | 3.4 | 2.9 | 2.5 | 3.1 | 2.4 | 1.5 | 1.6 |
| p-Xylene and m-Xylene | 33.2 | 48.6 | 51.0 | 51.1 | 53.6 | 52.2 | 56.5 |
| o-Xylene | 4.8 | 8.2 | 9.2 | 11.4 | 9.3 | 11.3 | 9.5 |
| Benzofuran | 4.3 | 2.2 | 1.0 | 1.0 | 0.6 | 0.4 | 0 |
| Indene | 2.6 | 2.4 | 3.0 | 3.8 | 8.5 | 11.6 | 13.6 |
| Phenol | 1.1 | 0.4 | 0.3 | 0.2 | 0.2 | 0 | 0 |
| Naphthalene | 7.7 | 4.4 | 4.5 | 3.5 | 2.8 | 0.6 | 0 |
| Olefin selectivity | | | | | | | |
| Ethylene | 54.9 | 37.9 | 30.9 | 23.2 | 19.6 | 16.1 | 18.0 |
| Propylene | 36.0 | 40.1 | 44.4 | 48.7 | 50.3 | 51.4 | 50.2 |
| Butenes | 7.3 | 19.9 | 21.3 | 24.9 | 26.4 | 27.6 | 25.9 |
| Butadience | 1.8 | 2.1 | 3.4 | 3.2 | 3.7 | 4.9 | 5.9 |

TABLE 5B
This table is a continuation of Table 5A. This table shows calculated values based on weighted
averages of the pure pine wood and methanol values.

| | H/C_{eff} (mol/mol) ratio | | | | |
|---|---|---|---|---|---|
| Compound | 0.47 | 0.76 | 1.05 | 1.25 | 1.64 |
| Pine wood WHSV (h⁻¹) | | | | | |
| Methanol WHSV (h⁻¹) | | | | | |
| Total WHSV (h⁻¹) | | | | | |
| Overall yields | | | | | |
| Aromatics | 7.0 | 8.0 | 8.8 | 9.4 | 10.1 |
| Olefins | 14.9 | 23.4 | 31.1 | 36.3 | 44.2 |
| Parrafins | 2.1 | 3.4 | 4.6 | 5.4 | 6.6 |
| Petrochemicals | 24.1 | 34.7 | 44.5 | 51.1 | 60.9 |
| Methane | 1.5 | 2.1 | 2.8 | 3.2 | 3.8 |
| CO₂ | 8.9 | 8.9 | 8.9 | 9.1 | 8.7 |
| CO | 16.5 | 13.9 | 11.6 | 10.4 | 7.1 |
| Coke | 32.9 | 26.0 | 19.7 | 16.2 | 7.8 |
| Total balance | 83.8 | 85.7 | 87.4 | 90.0 | 88.3 |
| Unidentified | 16.2 | 14.3 | 12.6 | 10.0 | 11.7 |
| Theoretical petrochemical yield | 71.2 | 76.6 | 82.2 | 85.8 | 93.2 |
| Aromatic selectivity | | | | | |
| Benzene | 9.1 | 7.8 | 6.6 | 6.0 | 4.3 |
| Toluene | 28.5 | 25.6 | 23.0 | 21.8 | 17.6 |
| Ethyl-benzene | 3.0 | 2.7 | 2.4 | 2.3 | 1.8 |
| p-Xylene and m-Xylene | 38.3 | 42.4 | 46.1 | 49.2 | 51.7 |
| o-Xylene | 5.8 | 6.7 | 7.4 | 8.0 | 8.6 |
| Benzofuran | 3.4 | 2.6 | 1.9 | 1.5 | 0.6 |
| Indene | 5.0 | 6.9 | 8.7 | 9.9 | 11.7 |
| Phenol | 0.9 | 0.7 | 0.5 | 0.4 | 0.2 |
| Naphthalene | 6.0 | 4.7 | 3.4 | 2.8 | 1.1 |
| Olefin selectivity | | | | | |
| Ethylene | 46.8 | 40.3 | 34.4 | 31.5 | 23.0 |
| Propylene | 39.1 | 41.6 | 43.9 | 46.0 | 46.9 |
| Butenes | 11.4 | 14.6 | 17.6 | 19.7 | 22.5 |
| Butadiene | 2.7 | 3.4 | 4.1 | 4.5 | 5.2 |

TABLE 5C
This table is a continuation of Tables 5A and 5B. This table shows the differences
between the experimental values in Table 5A and the calculated values in Table 5B.

| | H/C_{eff} (mol/mol) ratio | | | | |
|---|---|---|---|---|---|
| Compound | 0.47 | 0.76 | 1.05 | 1.25 | 1.64 |
| Pine wood WHSV (h⁻¹) | | | | | |
| Methanol WHSV (h⁻¹) | | | | | |
| Total WHSV (h⁻¹) | | | | | |
| Overall yields | | | | | |
| Aromatics | 7.8 | 10.8 | 12.6 | 11.7 | 3.9 |
| Olefins | −1.4 | −4.7 | −4.7 | −3.4 | 3.7 |
| Parrafins | −0.9 | −0.5 | −0.9 | −0.7 | −0.3 |
| Petrochemicals | 5.3 | 5.8 | 6.9 | 7.6 | 7.4 |
| Methane | −0.1 | 0.5 | −1.3 | −1.1 | −0.7 |
| CO₂ | −1.6 | −2.1 | −1.8 | −1.9 | −0.9 |
| CO | −1.6 | −1.3 | −2.2 | −2.7 | −1.3 |
| Coke | −2.6 | −2.6 | −1.9 | −1.7 | −0.4 |
| Total balance | −0.5 | 0.1 | −0.2 | 0.2 | 4.0 |
| Unidentified | 0.5 | −0.1 | 0.2 | −0.2 | −4.0 |
| Theoretical petrochemical yield | | | | | |
| Aromatic selectivity | | | | | |
| Benzene | −2.6 | −1.1 | −0.8 | −0.2 | 0.2 |
| Toluene | −3.9 | −3.8 | −2.8 | −4.9 | 0.4 |
| Ethyl-benzene | −0.1 | −0.2 | 0.7 | 0.1 | −0.3 |
| p-Xylene and m-Xylene | 10.3 | 8.6 | 5.0 | 4.4 | 0.5 |
| o-Xylene | 2.4 | 2.5 | 4.0 | 1.3 | 2.7 |
| Benzo furan | −1.2 | −1.6 | −0.9 | −0.9 | −0.2 |
| Indene | −2.6 | −3.9 | −4.9 | −1.4 | −0.1 |
| Phenol | −0.5 | −0.4 | −0.3 | −0.2 | −0.2 |
| Naphthalene | −1.6 | −0.2 | 0.1 | 0.0 | −0.5 |
| Olefin selectivity | | | | | |
| Ethylene | −8.9 | −9.4 | −11.2 | −11.9 | −6.9 |
| Propylene | 1.0 | 2.8 | 4.8 | 4.3 | 4.5 |
| Butenes | 8.5 | 6.7 | 7.3 | 6.7 | 5.1 |
| Butadiene | −0.6 | 0.0 | −0.9 | −0.8 | −0.3 |

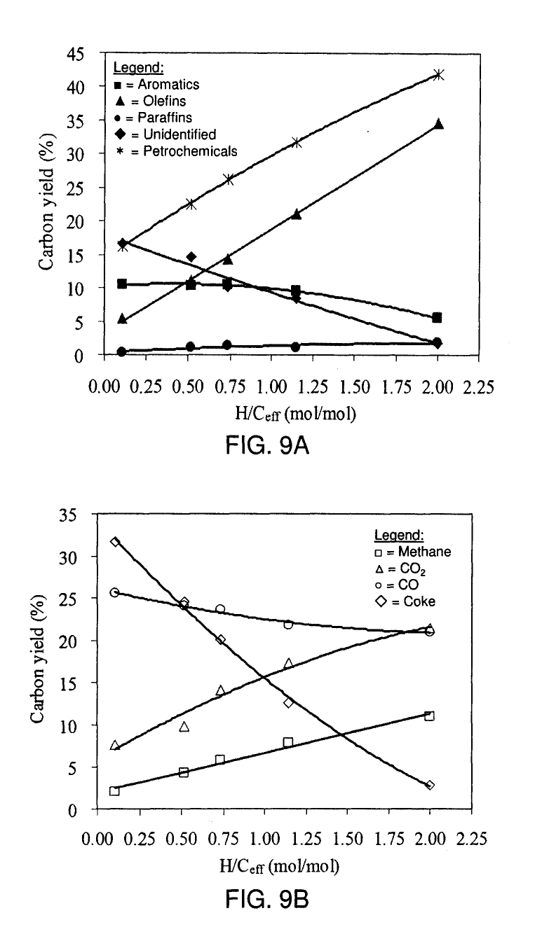

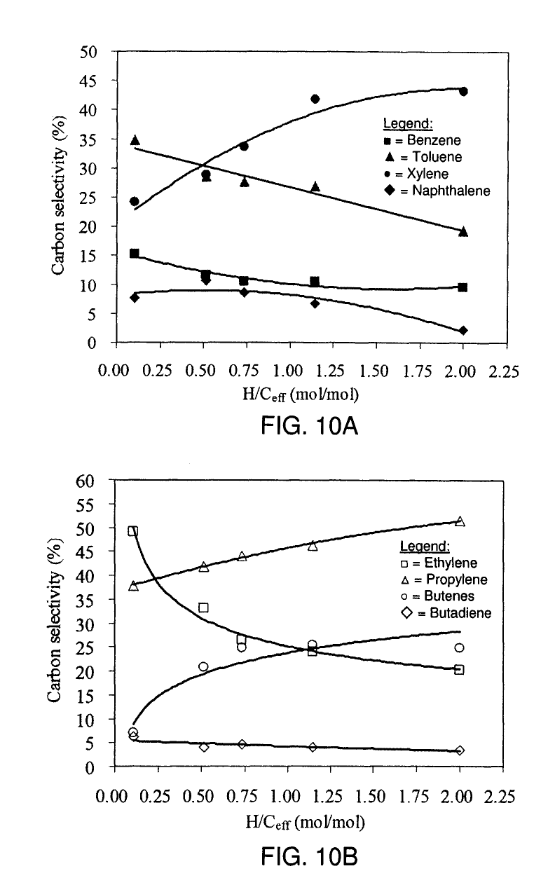

The effects of varying the hydrogen to carbon effective (H/C_{eff}) ratio at 500° C. are also investigated. The product carbon yields and selectivities as a function of H/Ceff ratio for co-CFP of pine wood and methanol at 500° C. are shown in FIGS. 9-10 and Table 6. Referring to FIG. 9, the unidentified compounds, CO, and coke yields decrease with an increase in the H/C_{eff} ratio, while olefins, CO₂, and methane yields increase significantly. Aromatic yield is relatively constant at around 10% as the H/C_{eff} ratio is increased from 0.11 to 1.15. The aromatic yield decreases to 5.5% with a further increase of the H/C_{eff} ratio to 2. Referring to FIG. 10, xylene selectivity increases with increasing H/C_{eff} ratio, and toluene selectivity decreases. Propylene selectivity increases with increasing H/C_{eff} ratio. Ethylene selectivity decreases non-linearly with an increase in H/C_{eff} ratio, whereas butenes selectivity shows the opposite trend and increases non-linearly. These non-linear curves indicate that the products are affected by the co-feeding, and a synergistic effect occurs at 500° C.

TABLE 6
Detailed product yields and selectivities for co-CFP of pine wood and methanol at various
H/C_{eff} ratios and 500° C. Aromatic selectivity is defined as the moles of carbon in the
product divided by the total moles aromatic carbon. Olefin selectivity is defined
as the moles of carbon in the product divided by the total moles olefin carbon.

| | H/C_{eff} ratio (mol/mol) | | | | |
|---|---|---|---|---|---|
| Compound | 0.11 | 0.51 | 0.74 | 1.15 | 2.00 |
| Pine wood WHSV (h⁻¹) | 0.41 | 0.24 | 0.28 | 0.18 | 0 |
| Methanol WHSV (h⁻¹) | 0 | 0.08 | 0.18 | 0.27 | 0.35 |
| Total WHSV (h⁻¹) | 0.41 | 0.33 | 0.46 | 0.44 | 0.35 |
| Overall yields | | | | | |
| Aromatics | 10.5 | 10.3 | 10.6 | 9.6 | 5.5 |
| Olefins | 5.4 | 11.1 | 13.5 | 21.1 | 34.6 |
| Parrafins | 0.3 | 1.2 | 1.3 | 1.1 | 1.8 |
| Petrochemicals | 16.2 | 22.6 | 25.4 | 31.8 | 41.9 |
| Methane | 2.1 | 4.3 | 5.5 | 8.0 | 11.0 |
| CO₂ | 7.7 | 9.8 | 14.1 | 17.3 | 21.5 |
| CO | 25.6 | 24.0 | 23.7 | 21.8 | 21.0 |
| Coke | 31.7 | 24.6 | 20.1 | 12.7 | 2.9 |
| Total balance | 83.3 | 85.3 | 88.7 | 91.5 | 98.3 |
| Unidentified | 16.7 | 14.7 | 8.0 | 8.5 | 1.7 |
| Theoretic petrochemical yield | 64.3 | 72.0 | 76.2 | 83.9 | 100 |
| Aromatic selectivity | | | | | |
| Benzene | 15.3 | 11.7 | 10.6 | 10.6 | 9.7 |
| Toluene | 34.7 | 28.5 | 27.7 | 26.9 | 19.3 |
| Ethyl-benzene | 4.5 | 2.2 | 2.3 | 1.9 | 2.5 |
| p-Xylene and m-Xylene | 24.1 | 28.8 | 33.6 | 41.8 | 43.3 |
| o-Xylene | 3.5 | 0.9 | 0.8 | 7.6 | 7.7 |
| Styrene | 3.7 | 5.2 | 6.2 | 0 | 4.4 |
| Benzofuran | 1.6 | 9.0 | 7.7 | 2.8 | 0 |
| Indene | 3.3 | 2.0 | 1.7 | 1.2 | 11.0 |
| Phenol | 1.5 | 1.0 | 0.8 | 0.5 | 0 |
| Naphthalene | 7.7 | 10.8 | 8.6 | 6.7 | 2.2 |
| Olefin selectivity | | | | | |
| Ethylene | 49.0 | 33.2 | 26.5 | 24.1 | 20.2 |
| Propylene | 37.8 | 41.9 | 44.1 | 46.3 | 51.5 |
| Butenes | 7.0 | 20.9 | 24.8 | 25.5 | 24.9 |
| Butadiene | 6.2 | 4.0 | 4.6 | 4.1 | 3.4 |

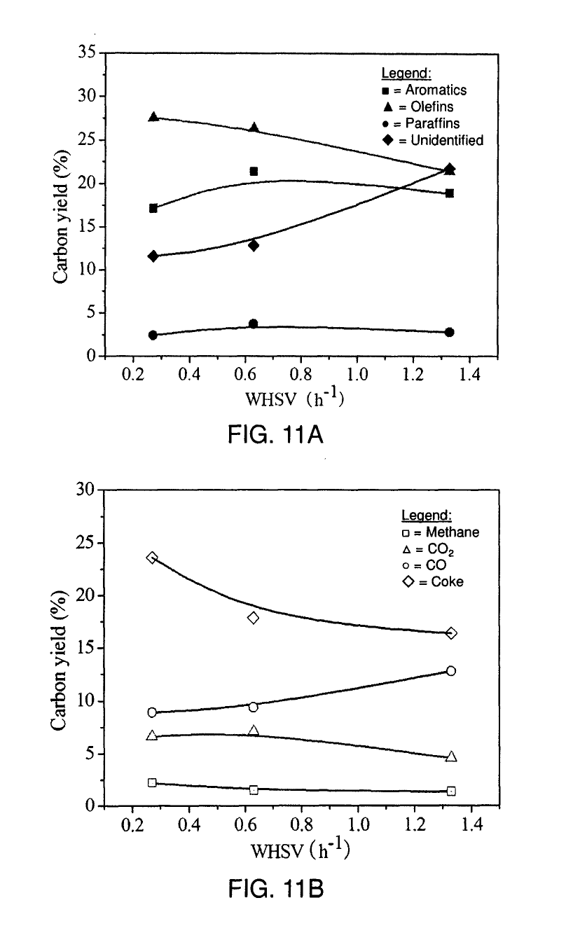

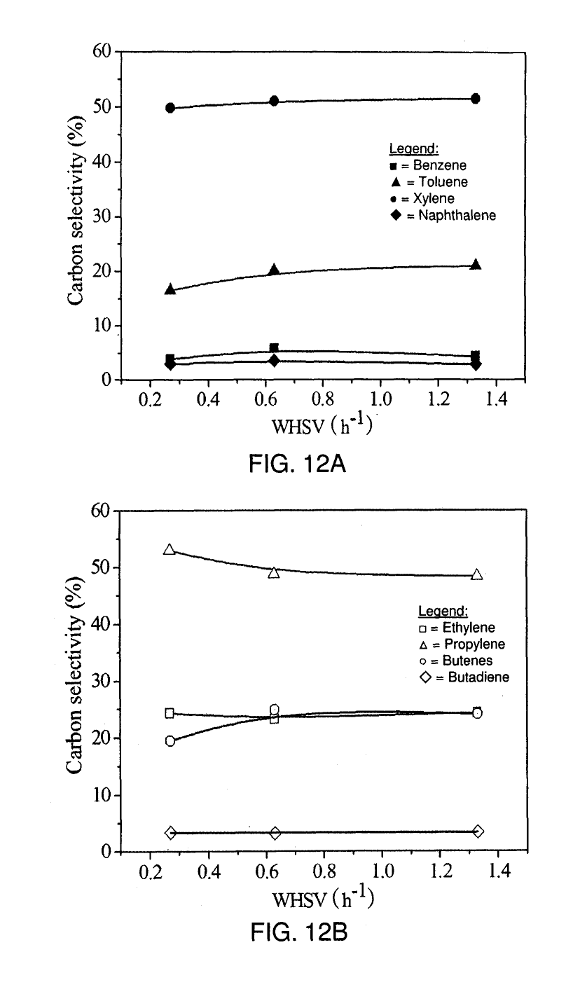

The effects of varying the total space velocity at a constant H/C_{eff} ratio is also investigated. FIGS. 11-12 show the product carbon yields and selectivities of co-CFP of pine wood and methanol at different total WHSV with a constant H/C_{eff} ratio of 1.05 at 450° C. Referring to FIG. 11, lower total WHSV favors olefins and coke production, while higher WHSV produces more CO and unidentified oxygenates. The aromatic and total petrochemical yields exhibit maximum values of 21.4% and 51.4%, respectively, at a WHSV of 0.63 h⁻¹. The aromatic and olefin selectivities were relatively constant over the WHSV range tested, with the exception of toluene and butenes, which increase slightly with WHSV. Table 7 shows the detailed product carbon yields and selectivities for co-CFP of pine wood and methanol at different total space velocities.

TABLE 7
Detailed product yields and selectivities for co-CFP of pine wood and
methanol at various total space velocities, H/C_{eff} = 1.05 and
450° C. Aromatic selectivity is defined as the moles
of carbon in the product divided by the total moles aromatic carbon.
Olefin selectivity is defined as the moles of carbon
in the product divided by the total moles olefin carbon.

| | Total WHSV ((h⁻¹) | | |
|---|---|---|---|
| Compound | 0.27 | 0.63 | 1.33 |
| Temperature (° C.) | 450 | 450 | 450 |
| Pine wood WHSV (h⁻¹) | 0.12 | 0.28 | 0.65 |
| WHSV of co-feeding alcohol (h⁻¹) | 0.15 | 0.35 | 0.68 |
| H/C_{eff} (mol/mol) | 1.05 | 1.05 | 0.98 |
| Overall yields | | | |
| Aromatics | 17.1 | 21.4 | 18.9 |
| Olefins | 27.6 | 26.3 | 21.5 |
| Parrafins | 2.4 | 3.7 | 2.8 |
| Petrochemicals | 47.1 | 51.4 | 43.2 |
| Methane | 2.2 | 1.5 | 1.3 |
| CO₂ | 6.6 | 7.1 | 4.6 |
| CO | 8.9 | 9.4 | 12.8 |
| Coke | 23.6 | 17.8 | 16.4 |
| Total balance | 88.4 | 87.2 | 78.2 |
| Unidentified | 11.6 | 12.8 | 21.8 |
| Theoretic petrochemical yield | 82.2 | 82.2 | 80.8 |
| Aromatic selectivity | | | |
| Benzene | 3.8 | 5.8 | 4.3 |
| Toluene | 16.5 | 20.2 | 21.0 |
| Ethyl-benzene | 1.8 | 3.1 | 3.6 |
| p-Xylene and m-Xylene | 49.8 | 51.1 | 51.6 |
| o-Xylene | 9.6 | 11.4 | 9.0 |
| Benzofuran | 1.8 | 1.0 | 0.5 |
| Indene | 13.2 | 3.8 | 7.0 |
| Phenol | 0.5 | 0.2 | 0.2 |
| Naphthalene | 3.0 | 3.5 | 2.9 |
| Olefin selectivity | | | |
| Ethylene | 24.3 | 23.2 | 24.3 |
| Propylene | 52.9 | 48.7 | 48.3 |
| Butenes | 19.5 | 24.9 | 24.1 |
| Butadiene | 3.3 | 3.2 | 3.3 |

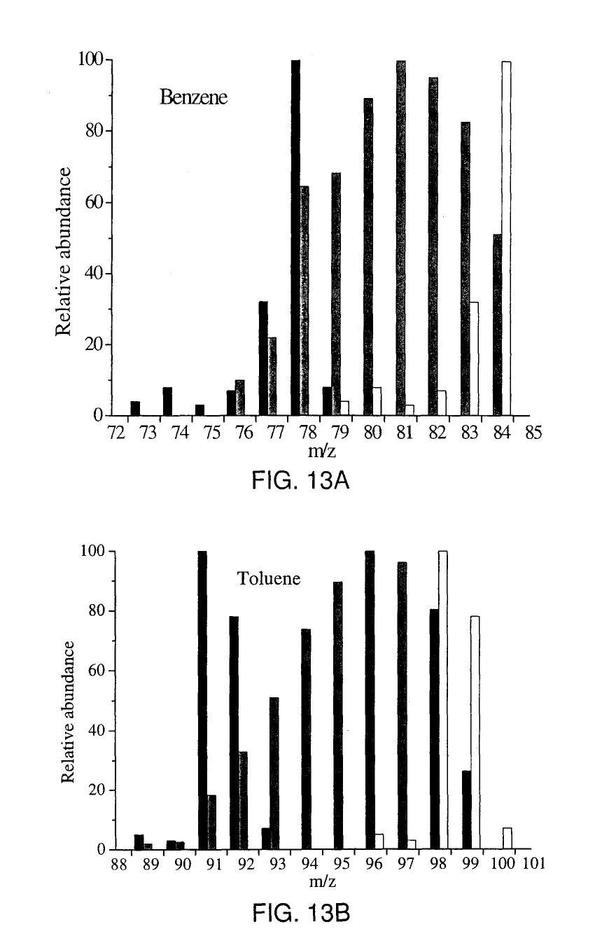

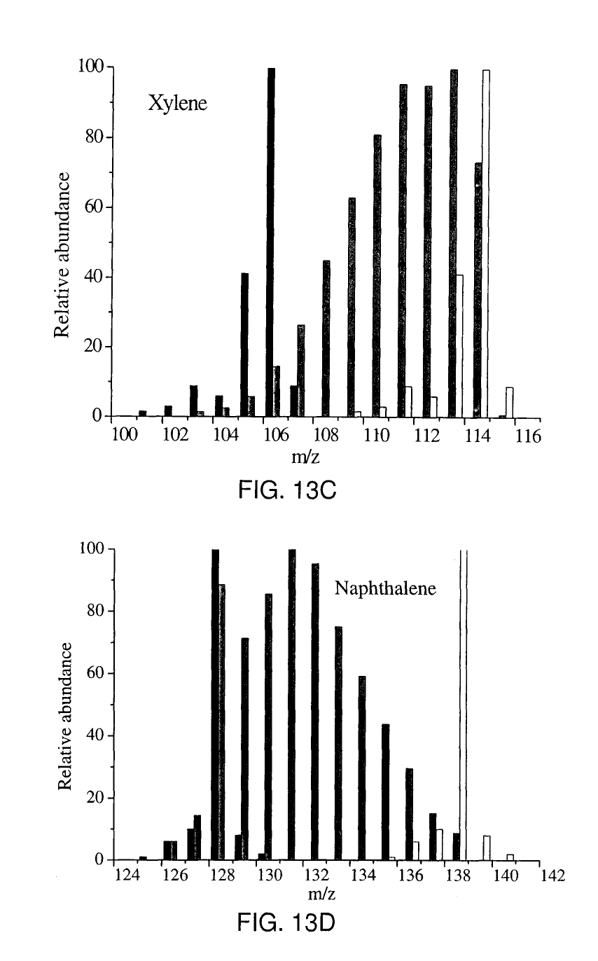

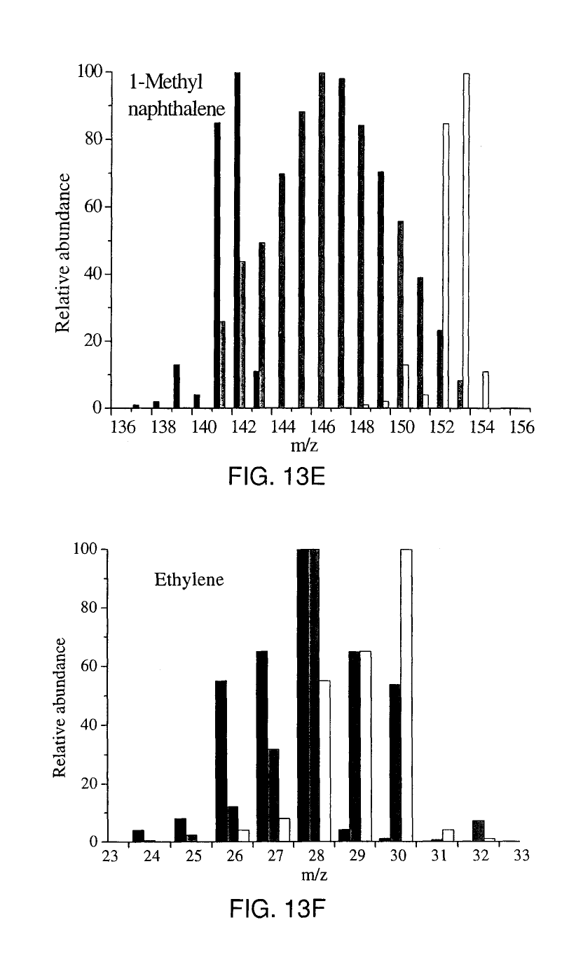

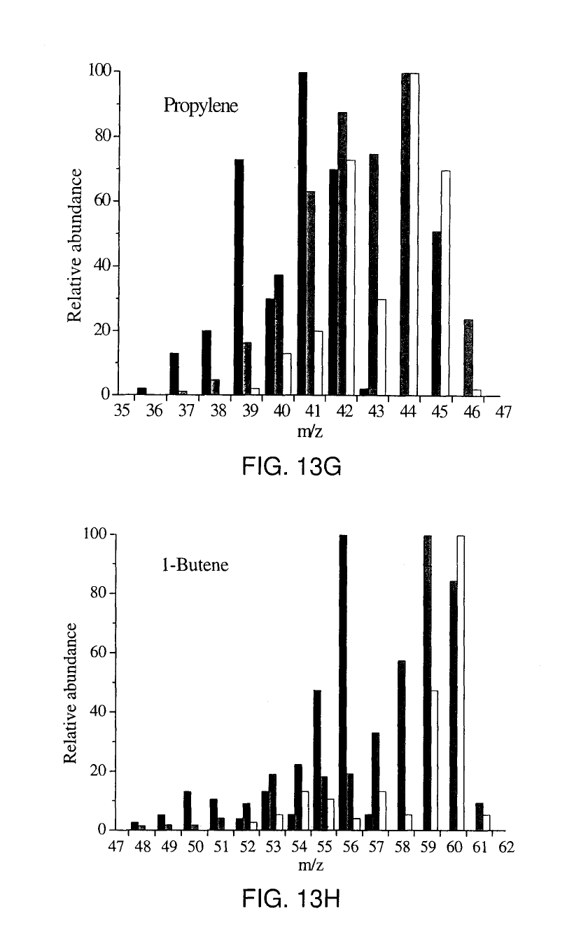

Isotopic labelling studies of co-catalytic fast pyrolysis of pine wood and methanol are also conducted. Co-CFP of ¹²C pine wood and isotopically labeled ¹³C methanol are conducted at 450° C. to determine how methanol enters the hydrocarbon pool. The WHSV values of ¹²C pine wood and ¹³C methanol are 0.30 h⁻¹ and 0.29 h⁻¹, respectively. The H/C_{eff} ratio of the mixture is 0.97. The helium fluidizing flow rate is 1200 mL min⁻¹ and the reaction time is 30 min. The mass spectra of the most abundant products are shown in FIG. 13. The fragmentation patterns for the pure ¹²C or pure ¹³C compounds are shown in black and white, respectively. The spectra of the products obtained during the co-feeding experiment are shown in grey. The results show that all the main products are a mixture of ¹²C- and ¹³C-labeled carbons. The distribution of carbon in benzene is a random mixture of the ¹²C and ¹³C carbons. However, the distributions of carbon within the other aromatics show trends. The distribution of toluene and xylene are both shifted to higher masses than would be expected from a random mixture of ¹²C- and ¹³C-labeled carbons. It is believed that this indicates that a random distributed benzene molecule is alkylated by a ¹³C-containing radical derived from ¹³C-labeled methanol preferably over a ¹²C-containing radical. Naphthalene shows the opposite trend as its spectrum is shifted toward lower masses than would be expected from a random mixture of ¹²C- and ¹³C-labeled carbons. It is believed that this indicates that the rate of naphthalene formation from the pine wood is higher than that from the methanol. The methyl naphthalene is not as shifted as the naphthalene, which indicates that the methyl group probably also comes from ¹³C-labeled methanol, similar to toluene and xylene. The olefin compounds spectra also show trends. When taking into account the overlap of the fragmentation peaks, the ethylene appears to be composed of more ¹²C than ¹³C carbon, while propylene and butenes show more ¹³C carbon. In summary, ¹²C and ¹³C are distributed in all of the hydrocarbon product molecules. Benzene is a random mixture of the ¹²C and ¹³C carbons, whereas naphthalene is formed much faster from the carbon of pine wood than methanol. However, their alkylated products are alkylated by a ¹³C-containing radical preferably over a ¹²C-containing radical. This may indicate that the methanol enters a zeolite catalytic process of biomass and that it is feasible to use feeds with high H/C_{eff} ratio to provide hydrogen to the hydrocarbon pool for biomass conversion.

### Co-Catalytic Fast Pyrolysis of Other Alcohols

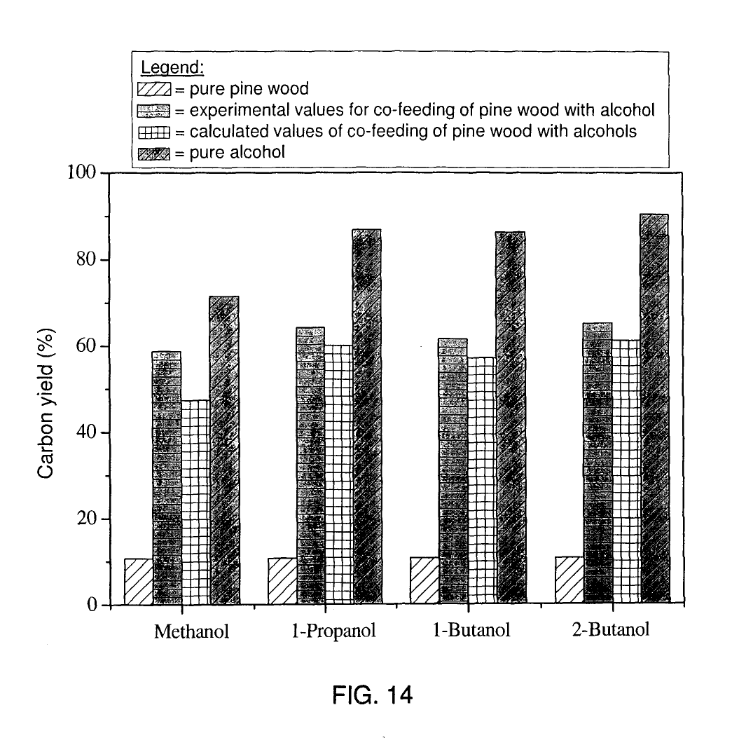

Co-catalytic fast pyrolysis of other alcohols (including 1-propanol, 1-butanol and 2-butanol) is also performed. FIG. 14 shows the petrochemical yield of co-CFP of pine wood and other alcohols at H/C_{eff}=1.25 and 450° C. The “calculated” values for the mixtures are found by a weighted average of the yields from CFP of pine wood and the alcohol separately. As shown in FIG. 14, CFP of pine wood produces 10.7% hydrocarbon yield, while methanol, 1-propanol, 1-butanol and 2-butanol yield is 71.1%, 86.8%, 86.3% and 90.3% of hydrocarbons, respectively. Co-feeding of pine wood and methanol yields the lowest amount of petrochemicals (58.8%), while co-feeding of pine wood and 2-butanol produces the highest carbon yield of 65.2%. However, compared with their calculated values, co-CFP of pine wood and methanol gives the highest increase of hydrocarbon yield and the best synergistic effect. The product carbon yields and selectivities of co-CFP of pine wood and the various alcohols at H/C_{eff}=1.25 and 450° C. are listed in Table 8. As shown in Table 8, the product selectivities of co-CFP of pine wood and 1-propanol, 1-butanol, and 2-butanol are very similar, but there is a difference with co-feeding pine wood and methanol, especially with respect to aromatic selectivities. Co-conversion of pine wood and methanol gives 62.9%, 5.8%, and 16.9% selectivities of xylene, benzene and toluene, respectively. Co-conversion of pine wood with other alcohols gives about 39.2-40.2%, 10.4-11.0%, and 38.6-39.3% of xylene, benzene and toluene, respectively. It is believed that is was due to methanol producing more methyl radicals than other alcohols at the same H/C_{eff} ratio, and that therefore, more benzene and toluene molecules are alkylated to xylene molecules.

TABLE 8
Detailed product yields and selectivities for CFP of pine wood and methanol, 1-propanol,
1-butanol and 2-butanol at H/C_{eft} = 1.25 and 450° C. Aromatic selectivity is defined as the
moles of carbon in the product divided by the total moles aromatic carbon. Olefin selectivity
is defined as the moles of carbon in the product divided by the total moles olefin carbon.

| | Co-feeding alcohol | | | | | | | | |
|---|---|---|---|---|---|---|---|---|---|
| Compound | Pine wood | Methanol | | 1-Propanol | | 1-Butanol | | 2-Butanol | |
| Pine wood WHSV (h⁻¹) | 0.35 | 0 | 0.20 | 0 | 0.24 | 0 | 0.30 | 0 | 0.29 |
| WHSV of co-feeding alcohol (h⁻¹) | 0 | 0.35 | 0.36 | 0.34 | 0.34 | 0.34 | 0.34 | 0.35 | 0.35 |
| Total WHSV (h⁻¹) | 0.35 | 0.35 | 0.56 | 0.34 | 0.58 | 0.34 | 0.64 | 0.35 | 0.64 |
| H/C_{eff} ratio (mol/mol) | 0.11 | 2.00 | 1.25 | 2.00 | 1.34 | 2.00 | 1.27 | 2.00 | 1.31 |
| Overall yields | | | | | | | | | |
| Aromatics | 5.9 | 11.1 | 21.1 | 13.3 | 16.3 | 15.2 | 17.2 | 15.2 | 15.6 |
| Olefins | 4.3 | 52.6 | 32.9 | 66.2 | 43.3 | 60.7 | 38.4 | 67.2 | 44.6 |
| Parrafins | 0.5 | 7.9 | 4.7 | 7.3 | 4.6 | 10.3 | 6.0 | 7.9 | 5.0 |
| Petrochemicals | 10.7 | 71.5 | 58.7 | 86.8 | 64.3 | 86.3 | 61.5 | 90.3 | 65.2 |
| Methane | 0.6 | 4.5 | 2.1 | 0.2 | 0.4 | 0.2 | 0.4 | 0.2 | 0.4 |
| CO₂ | 8.9 | 8.9 | 7.2 | 0.2 | 2.3 | 0.2 | 2.2 | 0.2 | 2.8 |
| CO | 19.7 | 5.1 | 7.7 | 0.4 | 6.5 | 1.1 | 6.9 | 0.5 | 7.6 |
| Coke | 41.6 | 2.1 | 14.5 | 1.6 | 10.7 | 1.0 | 14.3 | 1.0 | 12.8 |
| Total Balance | 81.4 | 92.1 | 90.2 | 89.2 | 84.1 | 88.7 | 85.5 | 92.1 | 88.8 |
| Theoretic (Toluene) | 61.3 | 100 | 85.8 | 100 | 87.5 | 100 | 86.2 | 100 | 86.9 |
| Experimental/theoretic | 16.6 | 71.5 | 68.4 | 86.8 | 73.5 | 86.3 | 71.4 | 90.3 | 75.0 |
| Aromatic selectivity | | | | | | | | | |
| Benzene | 10.8 | 3.3 | 5.8 | 11.8 | 11.0 | 11.5 | 10.6 | 12.0 | 10.4 |
| Toluene | 32.2 | 15.6 | 16.9 | 43.1 | 39.3 | 40.8 | 38.7 | 43.8 | 38.6 |
| Ethyl-benzene | 3.4 | 1.6 | 2.4 | 3.6 | 3.7 | 3.5 | 4.2 | 3.7 | 4.2 |
| p-Xylene and m-Xylene | 33.2 | 56.5 | 53.6 | 31.0 | 32.8 | 27.9 | 34.2 | 30.5 | 34.0 |
| o-Xylene | 4.8 | 9.5 | 9.3 | 7.0 | 6.4 | 6.0 | 6.0 | 6.7 | 6.2 |
| Benzofuran | 4.3 | 0 | 0.6 | 0.1 | 0.5 | 0.1 | 0.5 | 0.1 | 0.4 |
| Indene | 2.6 | 13.6 | 8.5 | 2.5 | 2.3 | 2.0 | 2.5 | 2.3 | 2.6 |
| Phenol | 1.1 | 0 | 0.2 | 0.2 | 0.5 | 0.1 | 0.2 | 0.2 | 0.5 |
| Naphthalene | 7.7 | 0 | 2.8 | 0.8 | 3.6 | 0.6 | 3.1 | 0.7 | 3.1 |
| Olefin selectivity | | | | | | | | | |
| Ethylene | 54.9 | 18.0 | 19.5 | 10.9 | 12.8 | 12.2 | 12.4 | 10.0 | 13.4 |
| Propylene | 36.0 | 50.2 | 50.3 | 55.7 | 54.5 | 52.7 | 53.8 | 53.8 | 51.5 |
| Butenes | 7.3 | 25.9 | 26.5 | 29.6 | 28.6 | 31.4 | 29.8 | 32.6 | 30.9 |
| Butadiene | 1.8 | 5.9 | 3.7 | 3.8 | 4.1 | 3.7 | 4.0 | 3.6 | 4.2 |

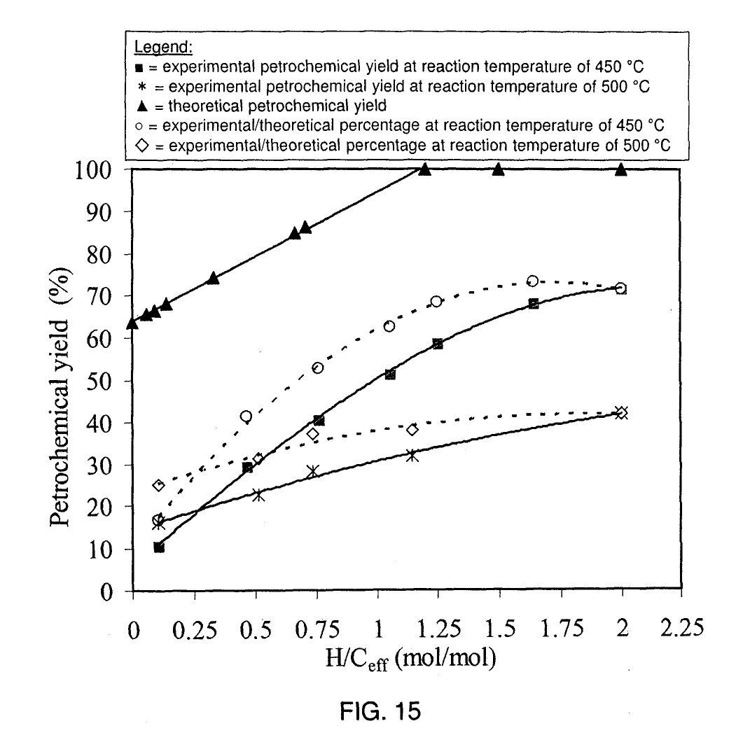

FIG. 15 shows the petrochemical yield as a function of H/C_{eff} ratio for co-CFP of pine wood and methanol at 450° C. and 500° C. As shown in FIG. 15, co-CFP of pine wood and methanol at 450° C. produces much more petrochemical product than co-CFP of pine wood and methanol at 500° C., and the gap increases with increasing H/C_{eff} ratio. The theoretical yields of dry pine wood and methanol are calculated by assuming toluene as the reaction hydrocarbon product. The equations used to calculate the theoretical yield are as follows:

[symbol]

The theoretical yield of dry pine wood is about 67% according to Equation 3, while that of methanol is 100%, as shown in Equation 4. The pine wood used in this example contains about 4% moisture; thus, the theoretical yield of pine wood based on the feed is 64.3%. The “theoretical petrochemical yield” plotted in FIG. 15 is drawn according to the theoretical petrochemical yield of ten biomass-derived feedstocks with different H/C_{eff} ratios, as described in H. Y. Zhang, Y. T. Cheng, T. P. Vispute, R. Xiao and G. W. Huber, Energy Environ. Sci., 2011, 4, 2297-2307, which is incorporated herein by reference. In FIG. 15, the “experimental/theoretical percentage” plots are calculated by dividing the experimental petrochemical yields by the theoretical petrochemical yield. As shown in FIG. 15, the experimental/theoretical value of the run at 450° C. increases non-linearly from 16.6% at H/C_{eff}=0.11 (corresponding to having only pine wood in the feed) to about 70% at H/C_{eff}=1.25, after which further increases in the H/C_{eff} ratio yields only a small change. This result illustrates that co-feeding wood and methanol produces much higher yields than if the two feed components are reacted separately and their products are mixed. Furthermore, an inflection point is observed at H/C_{eff} ratio=1.25, at which point, the increase in the petrochemical yield slows with an increase in H/C_{eff} ratio. This suggests that the use of an H/C_{eff} ratio of 1.25 is optimal, in some cases. In this set of embodiments, the benefits of adding additional methanol may be outweighed by increases in system cost.

While the invention has been explained in relation to various embodiments, it is to be understood that various modifications thereof will become apparent to those skilled in the art upon reading the specification. Therefore, it is to be understood that the invention disclosed herein includes any such modifications that may fall within the scope of the appended claims.

1. A method for producing one or more fluid hydrocarbon products from a solid hydrocarbonaceous material comprising:
feeding a first reactant comprising the solid hydrocarbonaceous material, and a non-solid second reactant comprising hydrogen or a source of hydrogen, to a reactor;
pyrolyzing within the reactor at least a portion of the first reactant under reaction conditions sufficient to produce one or more pyrolysis products; and
catalytically reacting at least a portion of the one or more pyrolysis products and at least a portion of the second reactant under reaction conditions sufficient to produce the one or more fluid hydrocarbon products.

2. The method of claim 1 wherein the reactor comprises a continuously stirred tank reactor, a batch reactor, a semi-batch reactor, a fixed bed reactor or a fluidized bed reactor.

3. The method of claim 1 wherein the reactor comprises a fluidized bed reactor.

4. The method of claim 1, wherein the first reactant comprises biomass.

5. The method of claim 1, wherein the first reactant comprises plastic waste, recycled plastics, agricultural solid waste, municipal solid waste, food waste, animal waste, carbohydrates, lignocellulosic materials, xylitol, glucose, cellobiose, hemi-cellulose, lignin, sugar cane bagasse, glucose, wood, corn stover, or a mixture of two or more thereof.

6. The method of claim 1 wherein the second reactant comprises molecular hydrogen or hydrogen that is covalently bonded to a non-hydrogen atom.

7. The method of claim 1 wherein the second reactant comprises H₂.

8. The method of claim 1 wherein the second reactant comprises an alcohol, ether, ester, carboxylic acid, aldehyde, ketone, hydrocarbon, or a mixture of two or more thereof.

9. The method of claim 1 wherein the second reactant comprises methanol, ethanol, propanol, butanol, or a mixture of two or more thereof.

10. The method of claim 1, wherein the first reactant and the second reactant comprise a feed for the reactor, the hydrogen to carbon effective ratio for the feed being in the range from about 0.75 to about 1.5, or from about 0.9 to about 1.5.

11. The method of claim 1, wherein the reactor is operated at a temperature in the range from about 400° C. to about 600° C.

12. The method of claim 1, wherein the solid hydrocarbonaceous material is fed to the reactor at a mass normalized space velocity of up to about 0.9 hour⁻¹.

13. The method of claim 1, wherein the catalytically reacting step is conducted in the presence of a catalyst, the catalyst comprising a zeolite catalyst comprising silica and alumina, the silica to alumina molar ratio being in the range from about 10:1 to about 50:1.

14. The method of claim 13, wherein the zeolite catalyst further comprises nickel, platinum, vanadium, palladium, manganese, cobalt, zinc, copper, chromium, gallium, an oxide of one or more thereof, or a mixture of two or more thereof.

15. The method of claim 13 wherein the catalyst comprises pores having a pore size from about 5 Angstroms to about 100 Angstroms.

16. The method of claim 1, further comprising the step of recovering the one or more fluid hydrocarbon products.

17. The method of claim 1, wherein the one or more fluid hydrocarbon products comprise aromatic compounds and/or olefin compounds.

18. The method of claim 1, wherein the one or more fluid hydrocarbon products comprise benzene, toluene, ethylbenzene, methylethylbenzene, trimethylbenzene, xylenes, indanes naphthalene, methylnaphthelene, dimethylnaphthalene, ethylnaphthalene, hydrindene, methylhydrindene, dimethylhydrindene, or a mixture of two or more thereof.

19. The method of claim 1, wherein the one or more fluid hydrocarbon products contain at least about 18 wt % aromatic compounds.

20. The method of claim 1, wherein during the catalytically reacting step a dehydration, decarbonylation, decarboxylation, isomerization, oligomerization and/or dehydrogenation reaction is conducted.

21. The method of claim 1, wherein the pyrolyzing step and the catalytically reacting steps are carried out in a single vessel.

22. The method of claim 1, wherein the pyrolyzing step and the catalytically reacting steps are carried out in separate vessels.

23. The method of claim 1, wherein the pyrolysis product is formed with less than about 30 wt % of the pyrolysis product being coke.

24. The method of claim 1, wherein the reactor is operated at a pressure of at least about 100 kPa.

25. The method of claim 1 wherein the reactor is operated at a pressure in the range from about 100 to about 600 kPa.

26. The method of claim 1 wherein the reactor is operated at a pressure below about 600 kPa.
